(19) United States
(12) Patent Application Publication
(10) Pub. No.: US 2011/0218253 A1
(43) Pub. Date: Sep. 8, 2011

(54) IMAGING-BASED IDENTIFICATION OF A NEUROLOGICAL DISEASE OR A NEUROLOGICAL DISORDER

(76) Inventors: Nicholas T. Lange, Cambridge, MA (US); Janet E. Lainhart, Salt Lake City, UT (US)

(21) Appl. No.: 13/014,471

(22) Filed: Jan. 26, 2011

Related U.S. Application Data
(60) Provisional application No. 61/298,477, filed on Jan. 26, 2010.

(51) Int. Cl. A61K 45/00; A61P 25/28; G06F 19/00

(52) U.S. Cl. 514/789; 702/19

(57) ABSTRACT

System(s) and method(s) are provided to enable imaging-based identification, detection, evaluation, and mapping of white matter microstructure and hemispheric organization of white matter in a central nervous system (CNS) structure afflicted by a neurological disease or disorder. Various embodiments exploit a set of diffusion tensor metrics to define a classification sub-space and determine a multivariate classifier through training data related to at least two groups of subjects: a first group of subjects afflicted by the neurological disease or neurological disorder, and a second group of subjects typically developing. The set of diffusion tensor metrics can be selected based at least on clinical information related to the neurological disease or neurological disorder and anatomy of CNS structure. Inclusion of tensor skewness asymmetry in such set yields an increase in sensitivity, specificity, accuracy, reliability, and predictive ability of the biological discrimination of subjects with and without a neurological disease or neurological disorder.

### CROSS-REFERENCE TO RELATED APPLICATIONS

This patent application claims priority to U.S. Provisional Patent Application Ser. No. 61/298,477 entitled “SYSTEMS AND METHODS FOR IDENTIFICATION OF NEUROLOGICAL DISEASES AND DISORDERS,” and filed on Jan. 26, 2010. The entirety of the above-captioned patent application is incorporated herein by reference.

### STATEMENT REGARDING FEDERALLY FUNDED RESEARCH

This invention was made with government support under the National Institute of Mental Health Grant Nos. MH084795 and MH080826. The government has certain rights in the invention.

### BACKGROUND

Autism is currently one of five disorders that fall under the umbrella of Pervasive Developmental Disorders (PDD), a category of neurological disorders characterized by severe and pervasive impairment in several areas of development. Autism is a complex developmental disability that typically appears during the first two years of life and affects the functioning of the brain, impacting development of social interaction and communication skills. Both children and adults on the autism spectrum typically show difficulties in verbal and non-verbal communication, social interactions, and leisure or play activities.

In February 2007, the Centers for Disease Control and Prevention issued their Autism and Developmental Disabilities Monitoring prevalence report. The report, which looked at a sample of 8 year olds in 2000 and 2002, concluded that the prevalence of autism had risen to 1 in every 150 American children, and almost 1 in every 94 boys. The issuance of this report caused a media uproar, but the news was not a surprise to the Autism Society or to the 1.5 million Americans living with the effects of autism spectrum disorder. Based on statistics from the U.S. Department of Education and other governmental agencies, autism is growing at a startling rate of 10-17 percent per year. At this rate, the Autism Society estimates that the prevalence of autism could reach 4 million Americans in the next decade.

Nonetheless, the spotlight shown on autism as a result of the prevalence increase opens opportunities for the nation to consider how to serve these families facing a lifetime of supports for their children. Currently, the Autism Society estimates that the lifetime cost of caring for a child with autism ranges from $3.5 million to $5 million, and that the United States is facing almost $90 billion annually in costs for autism. This figure includes research, insurance costs and non-covered expenses, Medicaid waivers for autism, educational spending, housing, transportation, employment, in addition to related therapeutic services and caregiver costs. Autism knows no racial, ethnic, or social boundaries, and can affect any family and any child.

### SUMMARY

One or more embodiments of the subject disclosure enable imaging-based identification, detection, evaluation, and mapping of white matter microstructure and hemispheric organization of white matter in a central nervous system (CNS) structure afflicted by a neurological disease or disorder. Various embodiments exploit a set of diffusion tensor metrics to define a classification sub-space and determine a multivariate classifier through training data related to at least two groups of subjects: a first group of subjects afflicted by the neurological disease or neurological disorder, and a second group of subjects typically developing. The set of diffusion tensor metrics can be selected based at least on clinical information related to the neurological disease or neurological disorder and anatomy of CNS structure. Inclusion of tensor skewness asymmetry in such set yields an increase in sensitivity, specificity, accuracy, reliability, and predictive ability of the biological discrimination of subjects with and without a neurological disease or neurological disorder.

In addition, alternative or additional embodiments of the subject disclosure allow determination of white matter microstructure (WMM) and hemispheric organization of white matter in various brain structures. Moreover, such additional or alternative embodiments enable utilization of asymmetries and atypicalities in the white matter microstructure and the hemispheric organization of white matter as biological indicators of neurological diseases and disorders.

In an aspect, tensor measures can be determined through diffusion tensor imaging (DTI). Diffusion tensor imaging is a magnetic resonance imaging (MRI) modality that can delineate white matter microstructure (WMM) based at least on orientation information of axons, and can provide valuable information regarding the pathology of various neurological diseases and disorders.

In another aspect, white matter microstructure and white matter hemispheric organization can be determined in the superior temporal gyrus and in the temporal stem, and atypicalities (as compared to a control subject, for example) in the WMM and the WM hemispheric organization can be utilized as indicators of a neurological disease or neurological disorder such as autism.

A description of embodiments of the subject disclosure and the advantages thereof will be set forth in part in the detailed description which follows, and in part will be obvious from the detailed description or may be learned by practice of the embodiments. Certain advantages of the subject disclosure and related embodiments can be realized and attained through various elements and combinations described herein or particularly pointed out in the appended claims. It is to be understood that both the foregoing general description and the following detailed description are exemplary and explanatory only and are not restrictive of the various embodiments of the subject disclosure.

### BRIEF DESCRIPTION OF THE DRAWINGS

The accompanying drawings, which are incorporated in and constitute a part of this specification, illustrate several example embodiments of the subject disclosure and serve to explain, at least in part, the various principles of the subject disclosure. Illustrative drawings set forth herein comprise:

FIG. 1 presents a high-level block diagram of an example system that enables identification of a neurological disease or neurological disorder in accordance with aspects described herein;

FIGS. 2A-2B illustrate, respectively, example segmentation of the superior temporal gyrus and temporal stem in diffusion tensor magnetic resonance images for raw and masked for white matter only in accordance with aspects described herein;

FIG. 3 illustrates diffusion tensor coefficients representative of diffusion tension metrics by group and hemisphere in accordance with aspects of the subject disclosure;

FIG. 4 illustrates an example tensor skewness (referred to SkewX), which represents interhemspheric oblate and prolate skewness) for example diffusion tensors having the same fractional anisotropy (FA);

FIG. 5 illustrates hemispheric asymmetry of STG tensor skewness in autism and typical development in accordance with aspects described herein;

FIG. 6 illustrates an example system that enables generation of a multivariate classifier that can supply a probability of a subject having a neurological disease or neurological disorder.

FIG. 7 depicts implementation of a multivariate classifier generated via the example system of FIG. 6 in accordance with aspects disclosed herein.

FIGS. 8A-8E illustrate example group classification boundaries for a group of four tensor metric pairs in accordance with aspects described herein;

FIG. 9 depicts a comparison of increased classification performance afforded by STG tensor skewness asymmetry and left STG FA in accordance with aspects described herein.

FIG. 10 illustrates an example system that enables integration of imaging-based prediction of a neurological disease or neurological disorder with clinical prediction thereof in accordance with aspects described herein;

FIG. 11 depicts longitudinal reduction of disparity amongst imaging-based posterior probability of disorder (PPD) and PPD that arises clinical assessment in accordance with aspects described herein;

FIG. 12 illustrates an example system that enables generation and processing of longitudinal data in accordance with aspects described herein; and

FIG. 13 illustrates a block diagram of an example operating environment that enables various features of the subject disclosure and performance of the various methods disclosed herein.

FIGS. 14-15 illustrate example methods for identifying a neurological disease or a neurological disorder in accordance with aspects described herein.

### DETAILED DESCRIPTION

Embodiments of the subject disclosure comprise systems and methods directed at detecting, evaluating, and mapping white matter microstructure and the hemispheric organization of white matter of a subject. The disclosed systems and methods can further comprise correlating white matter microstructure and the hemispheric organization of white matter with one or more neurological diseases or disorders such as autism.

As described herein, a “subject” can be an animal, e.g., a human being. A subject also can be a non-human animal. Examples of a non-human animal include but are not limited to a mouse, rat, marmot, pig, monkey, chimpanzee, orangutan, cat, dog, sheep, or cow. A subject can be a natural animal. A subject also can be a transgenic, non-human animal including but not limited to a transgenic mouse or transgenic rat.

Embodiments of the subject disclosure comprise systems and methods directed at detecting, evaluating, and mapping white matter microstructure and the hemispheric organization of white matter extracted from a subject. For example, the disclosed systems and methods can be applied to a brain or a spinal cord excised from a subject, e.g., an animal or transgenic animal. In some aspects, the disclosed systems and methods can be applied to a portion or segment of a subject's excised brain or spinal cord.

The disclosed systems and methods also can comprise identifying and developing biological determinants of neurological diseases and disorders. Such biological determinants can be directed at white matter microstructure or the hemispheric organization of white matter or both.

The disclosed systems and methods can further comprise correlating white matter microstructure or the hemispheric organization of white matter or both with one or more neurological diseases or disorders such as autism.

Disclosed herein are systems and methods for evaluating the clinical effects of treatment, which can be manifested by changes or improvements or both in white matter microstructure and the hemispheric organization of white matter.

While various aspects of the subject disclosure are illustrated in connection with diffusion tensor imaging based on magnetic resonance imaging (MRI), it should be appreciated that other imaging techniques, such as positron emission tomography (PET), various computed tomorgraphies (CTs), magnetic resonance spectroscopy (MRS), MRS imaging (MRSI), magnetoencephalography (MEG), or near infrared spectroscopy (NIRS), can be exploited to obtain a set of one or more diffusion tensor metrics. For instance, in certain scenarios, CNS structure afflicted by a specific neurological disease or disorder can be suited for time-dependent X-ray based imaging which can yield at least one tensor diffusion metric in the set of one or more diffusion metrics Likewise, autism is employed to exemplify aspects or features of the principles underlying the subject disclosure, which can be implemented for identification of any or most any neurological disease or neurological disorder.

Definitions and Nomenclature

At least part of the terminology used herein is for the purpose of describing particular, yet not exclusive, embodiments only and is not intended to be limiting.

As used in the specification and the appended claims or inventive concepts, the singular forms “a,” “an” and “the” can include plural referents unless the context clearly dictates otherwise. Thus, for example, reference to “a compound” includes mixtures of compounds, reference to “a pharmaceutical carrier” includes mixtures of two or more such carriers, and the like. Reference to “a component” can include a single or multiple components or a mixture of components unless the context clearly dictates otherwise.

Ranges may be expressed herein as from “about” one particular value, and/or to “about” another particular value. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

The word “or” as used herein means any one member of a particular list and also includes any combination of members of that list.

“Optional” or “optionally” means that the subsequently described event or circumstance may or may not occur, and that the description includes instances where said event or circumstance occurs and instances where it does not.

As employed in this specification and annexed drawings, the terms “unit,” “component,” “system,” “platform,” and the like are intended to include a computer-related entity or an entity related to an operational apparatus with one or more specific functionalities, wherein the computer-related entity or the entity related to the operational apparatus can be either hardware, a combination of hardware and software, software, or software in execution. One or more of such entities are also referred to as “functional elements.” As an example, a unit may be, but is not limited to being, a process running on a processor, a processor, an object, an executable computer program, a thread of execution, a program, a memory (e.g., a hard disc drive), and/or a computer. As another example, a unit can be an apparatus with specific functionality provided by mechanical parts operated by electric or electronic circuitry which is operated by a software or a firmware application executed by a processor, wherein the processor can be internal or external to the apparatus and executes at least a part of the software or firmware application. An illustration of such a unit can be magnetic resonance imaging equipment. In addition or in the alternative, a unit can provide specific functionality based on physical structure or specific arrangement of hardware elements; an illustration of such a unit can be a magnet or a material that emits X rays. As yet another example, a unit can be an apparatus that provides specific functionality through electronic functional elements without mechanical parts, the electronic functional elements can include a processor therein to execute software or firmware that provides at least in part the functionality of the electronic functional elements. An illustration of such apparatus can be control circuitry, such as a programmable logic controller. The foregoing example and related illustrations are but a few examples and are not intended to limiting. Moreover, while such illustrations are presented for a unit, the foregoing examples also apply to a component, a system, a platform, and the like. It is noted that the terms “unit,” “component,” “system,” “platform” can be utilized interchangeably

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed systems and methods. These and other components are disclosed herein, and it is understood that when combinations, subsets, interactions, groups, etc. of these components are disclosed that while specific reference of each various subject and collective combinations and permutation of these may not be explicitly disclosed, each is specifically contemplated and described herein, for all systems and methods. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The various embodiments of the subject disclosure can be understood more readily by reference to the following detailed description and the Examples included therein and to the Figures and their previous and following description.

As will be appreciated by one skilled in the art, the systems and methods may take the form of an entirely hardware embodiment, an entirely software embodiment, or an embodiment combining software and hardware aspects. Furthermore, the systems and methods may take the form of a computer program product on a computer-readable storage medium having computer-readable program instructions (e.g., computer software) embodied in the storage medium. More particularly, the present systems and methods may take the form of web-implemented computer software. Any suitable computer-readable storage medium may be utilized including hard disks, CD-ROMs, optical storage devices, or magnetic storage devices.

Embodiments of the systems and methods are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

Such computer-executable instructions (e.g., computer programming code instructions) also can be stored in a computer-readable storage medium (e.g., a memory) that, in response to execution by a processor, can direct a computer or other programmable data processing apparatus, such as a computing device, comprising the processor to function or operate in a particular manner, such that the computer-executable instructions stored in the computer-readable storage medium (e.g., the memory) produce an article of manufacture including computer-readable instructions for implementing the function(s) or operation(s) specified in the flowchart block or blocks. The computer-executable instructions also can be loaded onto a computer or other programmable data processing apparatus, such as a computing device, to cause a series of operational steps to be performed on the computer or the other programmable apparatus to produce a computer-implemented process or method such that the computer-executable instructions that execute on the computer or the other programmable apparatus provide steps for implementing the function(s) or operation(s) specified in the flowchart block or blocks.

Accordingly, in an aspect, blocks of the block diagrams and flowchart illustrations support combinations of means for performing the specified functions, combinations of steps for performing the specified functions and program instruction means for performing the specified functions. It should be appreciated that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

The brain and spinal cord include gray matter connected by channels of white matter, sometimes referred to as fiber bundles, or fasciculi. As referred to herein, “white matter” generally refers to bundles of myelinated nerve cell processes (or axons), which connect various gray matter areas (the locations of nerve cell bodies) of the brain and spinal cord to each other, and carry nerve impulses between neurons.

The term “myelin” refers to an adaptation of the vertebrate nervous system that is essential for the fast propagation of action potentials along axons. In myelinated axons, current quickly passes between the nodes of Ranvier, which are unmyelinated regions of the axon that contain the voltage-gated sodium channels that propagate the action potential. In the central nervous system (CNS), oligodendrocytes form myelin, whereas Schwann cells fulfill this function in the peripheral nervous system (PNS). These specialized glial cells repeatedly wrap their membranes around axons to form a highly compacted, multilayered myelin sheath composed of specific proteins and lipids. The importance of myelin is demonstrated by a number of pathological conditions in humans, in which disruption of myelin affects conduction and health of CNS and PNS axons, leading to debilitating neurological deficits. For example, disruption of myelinated axons causes human diseases, including multiple sclerosis and Charcot-Marie-Tooth peripheral neuropathies.

As used herein, “neurological disease” or “neurological disorder” refers to diseases, disorders, and conditions that affect neurological functions and structures including, but not limited to, autism spectrum disorders, autism, Fragile X, seizure, tuberous sclerosis, aphasia, Parkinson's disease, Wilson's disease, amyotrophic lateral sclerosis, Alzheimer's disease, coma, epilepsy, attention-deficit hyperactivity disorder (ADHD), stroke, depression, multiple sclerosis, schizophrenia, addiction, neurogenic pain, cognitive/memory dysfunction, obsessive compulsive disorders, dementia, traumatic brain injury, post-traumatic stress disorder (PTSD), coma, minimally conscious or vegetative states, locked-in syndrome, spinal cord injury, peripheral neuropathy, migraine, epilepsy, brain tumors, spinal tumors, and any other biological brain or central nervous system disorder. A subject affected by such a neurological disease and disorder can exhibit a neuropathology that includes asymmetries and atypicalities in the white matter microstructure or the hemispheric organization of white matter or both.

To identify the paths followed by the white matter and to detect differences between functional and dysfunctional brains and other members of the central nervous system (CNS), clinicians and researchers generally utilize diffusion tensor magnetic resonance imaging (referred to herein as DT-MRI or DTI) to observe the diffusion of water in brain tissue. Using those observations, one can extract (e.g., infer) value(s) of the diffusion tensor at different locations in such tissue and their diffusion pathways. A physiological characteristic of the white matter tracks is that water tends to diffuse anisotropically in the direction of those tracks. Thus, by observing the directions in which water diffuses at different locations in the brain and spinal cord, one can identify the directions of major fiber bundles within the brain and CNS. The preferred direction of diffusion, and the extent of that preference, can be described by a tensor field made up of diffusion tensors with each diffusion tensor being associated with a different location in the brain. Thus, if one could evaluate the diffusion tensor at each point in the brain and CNS, one would be able to determine the directions of the fiber bundles in that tissue. Systems and methods of estimating a value of a diffusion tensor using DT-MRI data are disclosed in U.S. Pat. Nos. 7,268,551, 7,570,049, 5,539,310, and 7,643,853, each of which are fully incorporated herein by reference.

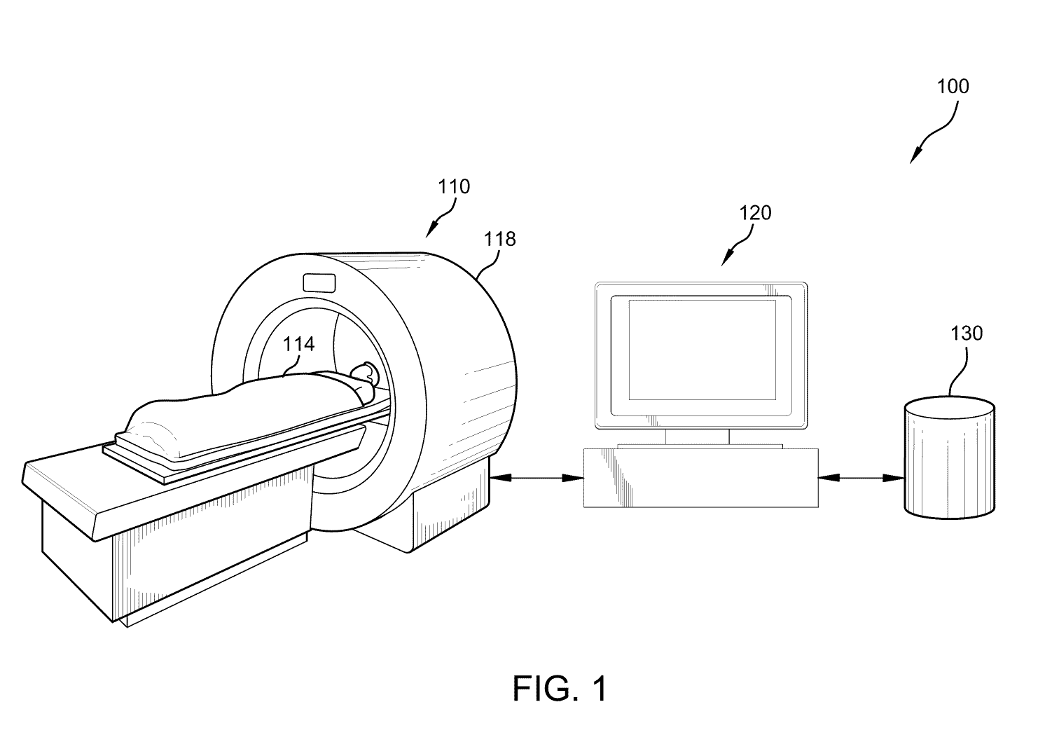

The systems and methods disclosed herein can be performed using various types magnetic resonance imaging machines, including “open” and “closed” MRIs. Generally, the standard “closed” MRI is performed in a narrow tube-like device about 2 feet in diameter and 6 feet to 8 feet long to optimize the images. Because of the small bore of the magnet, some subjects can experience claustrophobia and have difficulty in cooperating during imaging. In the “open” type, the large magnet that generates the image is generally suspended a couple of feet above the patient, and except for its supports, the unit is open all around. If a subject is severely claustrophobic or obese or morbidly obese (e.g., over 300 pounds in weight), a clinician (e.g., a doctor) performing an imaging procedure of such subject can suggest that the imaging procedure be conducted in an “open” MRI unit because it has more room inside than a closed magnet. For example, a skilled person in the art is familiar with various MRI instruments, the magnetic strength of various MRI instruments, and the manufacturers of various MRI instruments. FIG. 1 illustrates a high-level block diagram of an example system 100 that enables identification of a neurological disease or neurological disorder in accordance with aspects described herein. In example system 100, imaging unit 110 probes nervous tissue of a subject 114 and generates imaging data (e.g., DTI data; not shown). In certain embodiments, imaging unit 110 represents a closed MRI machine comprising a magnet 118 and related circuitry (not shown) that enables acquisition of imaging data (e.g., collection of imaging data and delivery or storage thereof). The imaging data is supplied to a computing unit 120, which can process (format, display, analyze, etc.) the imaging data. Computing unit 120 can convey (render or display, transmit, etc.) raw imaging data or processed imaging data, such as a diffusion tensor fields, a diffusion tensor metric, or the like. In an aspect, computing unit 120 can supply raw imaging data or processed imaging data to a data storage unit 130, which can be embodied in a computer-readable storage medium or a logic element (register, file, database, etc.) encoded therein. Exchange of information (e.g., data, signaling or instructions, or the like) amongst computing unit 120 and imaging unit 110 or computing unit 120 and data storage unit 120 can be accomplished wirelessly or via wireline communication.

Diffusion weighted imaging probes non-invasively the restriction of random Brownian motion of tissue water and provides clues about the microenvironment in which the water molecules are dispersing. Neurological disease or neurological disorder can change, directly or indirectly, the diffusion characteristics of the underlying tissue and can therefore be detected using diffusion weighted imaging techniques. For example, the failure of the sodium potassium ATP pump causes cytotoxic edema following acute brain infarction. This process shifts extracellular fluid into the intracellular space in the affected area showing changes in the signal intensities on MR images as compared to the unaffected brain tissue.

In the brain or other nervous fibers, water cannot diffuse as freely in all directions as surrounding tissue structures limit their mobility, hence creating preferred directions of diffusion. For example, it is easier for water to diffuse along the length of a white matter fiber rather than across the fiber. This property, known as diffusion anisotropy, is physically linked to the anisotropy of the tissue structure. Standard diffusion weighted imaging (DWI) acquires data in three orthogonal planes, for example, a first plane with a normal along a first direction, such as \hat{x}=[100], a second plane with a normal along a second direction, such as ŷ=[010], and a third plane with a normal along a third direction, such as \hat{z}=[001]. The signal intensity of diffusion weighted images depends on the pulse sequence used, the T2 magnetic resonance of the underlying tissue, and the diffusion characteristics of that tissue. In certain embodiments, because of such complexity, DWI can be difficult to interpret in conditions where the underlying T2 is altered. In certain embodiments, computing unit 120 also can control implementation of the pulse sequence through delivery of control instruction(s) to imaging unit 110 which, in response to reception of the control instruction(s), can implement the pulse sequence through adjustments of applied magnetic field produced in magnet 118.

A set of images called apparent diffusion coefficient (ADC) maps can elucidate certain aspects in such situations. ADC maps can be created by combining information from diffusion weighted images and information from images obtained from the same pulse sequence, but with low or no diffusion gradients “ON” (e.g, low magnetic field (B) value) to reduce the T2 contributions from tissue also called the “T2 shine thru effect.” Signal intensities of diffusion weighted images and ADC maps can be very different. A decrease in ADC causes increased signal intensity on DWI, but decreased signal intensity on ADC maps. In certain embodiments, computing unit 120 can generate at least one ADC map, and can retained the at least one ADC map in data storage unit 130 (also referred to as data storage 130 or data repository 130).

Diffusion, however, is a three-dimensional process in space. To acquire more detailed information about anisotropic diffusion properties of underlying tissue, simple standard DWI generally is not sufficient. While ADC maps can reveal the tendency of water molecules to diffuse within a voxel, directional variation is also required to image 3D anisotropic diffusion. One mathematical representation commonly utilized to model 3D anisotropy is a tensor. In an aspect, such tensor is a diffusion tensor represented by a 3×3 square symmetric matrix of coefficients (e.g., real numbers) of which only six (6) are unique. By sampling six (6) or more diffusion directions and establishing a relationship between the acquired data and applied diffusion gradients in a pulse sequence that serves as a probe, the directional variation in the tendency of water molecules to diffuse within a voxel can be imaged. Such technique is called diffusion tensor imaging (DTI). DTI generally describes local diffusion along each direction in a set of three orthogonal directions, and interactions between such orthogonal directions thus providing important information about tissue connectivity. DTI can be utilized, for example, to investigate white matter structure of the brain and changes that occur in association with the neurological disease process in vivo. DTI also can be employed to document integrity, displacement, or involvement of white matter tracts in different clinical conditions such as trauma or tumors and can predict outcome of treatment. Fiber tracking or tractography is the process of tracing the three-dimensional course of white matter fiber tracts using DTI data sets. Moreover, DTI can be utilized to further understand white matter pathways. Theoretically, the acquisition of imaging data along six (6) diffusion directions is sufficient to calculate diffusion tensor information in each voxel, yet the higher the number of directions the more robust the diffusion tensor calculation is, but the longer an imaging scan will take. Thus, there is generally a trade-off between data acquisition time and precision of estimated diffusion tensor. Typically, the number of diffusion directions employed to estimate the diffusion tensor from DTI data is of the order of about 6 to about 60. In an aspect, the number of diffusion directions is an imaging control parameter stored in computing unit 120 or data storage 130, and employed by imaging unit 110.

From the DTI data, several other diffusion maps or indices can be calculated. For example, commonly utilized indices are fractional anisotropy (FA), representing intra-voxel directional diffusion coherence; ADC (apparent diffusion coefficient), summarizing diffusion in a single direction; mean diffusivity (MD), summarizing diffusion in all directions; and axial diffusivity (D_{A}) and radial diffusivity (D_{R}), representing diffusion parallel and perpendicular to white matter fibers, respectively. Fractional Anisotropy (FA) relates to the level of directional organization of the tissue microstructure. In the developing brain for instance, changes in T1 and T2 signals are seen later than directional bias as measured via FA. In highly directional tissues such as white matter tracts or skeletal muscle one would expect a high FA value. In gray matter, a low FA value is measured since the tissue structure is not organized into fibers and is generally isotropic. A further level of detail of diffusion features in nervous tissue can be displayed by color-coding directionality on the FA map; in an embodiment, computing unit 120 can generate and render a FA map color-coded according to such directionality. Such color-coded images convey not only the location of large white matter tracts but also their prevalent direction using the diffusion tensor information.

Measurement of the diffusion coefficient from the DTI data of nervous tissue provides an indication of mobility of water or a fluid (e.g., liquid imaging contrast) in the nervous tissue. Large values of ADC are indicative of free or substantially free water or fluid, while smaller values generally indicate that mobility of water or fluid is constrained by the local tissue environment. Using the tensor model, the ADC is calculated as the trace of the diffusion tensor by averaging the diagonal elements of a 3×3 matrix representation of the diffusion tensor. Such calculation yields the mean diffusivity, or ADC in the tissue, which is independent of the diffusion encoding.

In the disclosed embodiments of the subject disclosure, a diffusion tensor field within a volume of nervous tissue, such as brain tissue, or spinal cord tissue, is characterized by a diffusion tensor ( [symbol] ) associated with each voxel in a non-empty set of voxels that span the volume of nervous tissue. The diffusion tensor [symbol] can be represented, in a reference system of three mutually orthogonal directions {\hat{x}, ŷ, \hat{z}}, by a 3×3 symmetric matrix of real numbers:

$$\overleftrightarrow{D} = [ \begin{matrix}{D}_{xx} & {D}_{xy} & {D}_{xz} \\ {D}_{xy} & {D}_{yy} & {D}_{yz} \\ {D}_{xz} & {D}_{yz} & {D}_{zz}\end{matrix} ] ,$$

Accordingly, as indicated hereinbefore, there are six (6) non-redundant matrix elements in the diffusion tensor: The three diagonal matrix elements Dₓₓ, D_{yy}, and D_{zz}, and the three off-diagonal matrix elements D_{xy}, D_{xz}, and D_{yz}. In an embodiment of example system 100, the group of diffusion tensors { [symbol] } associated with the non-empty set of voxels spanning the volume of tissue can be retained in data storage 130. In alternative or additional embodiments, such group can be retained in a computer-readable storage medium local to the computing unit 120.

Tractography typically includes estimating the values a group of diffusion tensors that compose the diffusion tensor field. The resulting estimate can be used to determine the orientation of white matter tracks in the brain.

Although the anatomy of the human brain has been studied extensively for over a century, many anatomical features of the human brain remain difficult to characterize. As an illustration, understanding of cortical structures remains is challenge primarily because cortical structures are extensively heterogeneous, both regionally and across subjects. White matter (WM) structures seem to share more common anatomical features across subjects at the deep white matter regions (DWM); there are many prominent axonal bundles that can be identified in all normal subjects at well-defined locations. However, the peripheral, more superficially located white matter (SWM), which fills the space between the DWM and the cortex, has not been well characterized in the past. For example, the SWM is known to contain short cortical association fibers, but their location, number, and trajectories are not sufficiently defined.

Such lack of anatomic knowledge about the SWM is understandable. The 3D axonal anatomy is, in general, difficult to understand by inspection of 2D histological sections. The entire WM of the adult human brain looks more or less homogeneous, both in myelin stained histological sections and in macroscopic slabs of native or fixed brains. The anatomy of very large fiber bundles can be studied by freezing and thawing repeatedly postmortem brains and subsequent manual peeling of fiber bundles. Such approach, however, cannot isolate smaller fiber bundles.

Embodiments of the subject disclosure comprise systems and methods directed at detecting, evaluating, and mapping white matter microstructure and the hemispheric organization of white matter. Such embodiments increase the accuracy, sensitivity, and reliability of the biological discrimination of subjects with and without neurological disorders or neurological diseases such as autism.

The disclosed systems and methods further comprise evaluating white matter microstructure and the hemispheric organization of white matter of a subject. Based on the results of the white matter microstructure, the disclosed systems and methods can further comprise identifying a subject for genetic screening or genetic testing. It is noted that in the subject disclosure the terms “genetic screening” and “genetic testing” are employed interchangeably. In the disclosed systems and methods, genetic screening can be performed to check for certain genes that potentially produce damaging changes, such as a neurological disease or disorder in a subject. The genetic screening can occur prior to, concomitant, or after the presentation of clinical symptoms.

Genetic screening is also useful to identify subjects that are carriers of a chromosomal abnormality or gene that can cause problems for either the offspring or the subject screened. For example, using the disclosed systems and methods, genetic screening can (1) confirm a diagnosis of a neurological disease or disorder if a subject has symptoms; (2) determine whether a subject is a carrier for a gene that can cause or exacerbate a neurological disease or disorder; (3) provide expectant subjects with information regarding whether an unborn offspring will have a neurological disease or neurological disorder; (4) determine whether a subject has an inherited disposition to a certain a neurological disease or disorder before symptoms start; and (5) evaluate the type or dose of a medicine or treatment that is most likely to effectively treat a neurological disease or disorder in a given subject. Thus, the types for genetic screening in the disclosed systems and methods can include, but are not limited to, diagnostic testing, predictive testing, presymptomatic testing, carrier testing, prenatal testing, and pharmacogenetic testing.

Using the disclosed systems and methods, associations between a gene and a neurological disease or disorder can be established by linkage studies. The skilled person can use polymorphic markers, which can be found in the population with a relatively high frequency, to identify relatives (e.g., siblings) that are affected by a disease or disorder. If during this study, one form of one marker (or of a close linked marker) is found significantly more often than expected by chance, then this marker is said to be close (or “linked”) to the disease-related gene. Thus, the disclosed systems and methods can help to elucidate the molecular genetics of neurological diseases and disorders such as autism.

Currently, the art recognizes that several genes can contribute to or cause mismyelination, demyelination, or dysmyelination. Mismyelination, demyelination, or dysmyelination can contribute to asymmetries or atypicalities in the white matter microstructure and the hemispheric organization of white matter that underlay neurological diseases and disorders. These genes include, but are not limited to, the following: myelin basic protein (MBP), Sonic hedgehog (Shh), Olig2, Notch, and Sox10.

For example, in the CNS, oligodendrocyte precursor cells arise in response to the signal Sonic hedgehog (Shh), which induces the expression of the bHLH transcription factor Olig2, the earliest known intrinsic regulator of oligodendrocyte specification. The Notch receptor, expressed by immature and maturing oligodendrocytes, may initially promote glial development but later interacts with axonally provided Jagged ligand to suppress oligodendrocyte maturation. In addition to its role in early events in Schwann cell development, Sox10 is also required for the terminal differentiation of oligodendrocytes.

The disclosed systems and methods can determine whether a subject demonstrates a potential intermediate brain imaging phenotype, which can be helpful in the discovery of new genes and other factors involved in the pathology of a neurological disease or disorder. Furthermore, the disclosed systems and methods also can comprise genetic screening to evaluate subjects with potential intermediate brain imaging phenotypes, and subjects who are carriers (e.g., subjects who have an abnormal gene for a neurological disease or disorder but who do not have any symptoms or visible evidence of the disease or disorder). Subjects can be carriers if the abnormal gene is recessive—that is, if two copies of the gene are needed to develop a disorder or disease. Carrier screening involves testing samples from subjects who do not have symptoms, but are at higher risk for carrying a recessive gene for a particular neurological disease or disorder.

The term “sample” can refer to a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g., a polypeptide or nucleic acid). A sample may also be any body fluid or excretion (for example, but not limited to, blood, urine, stool, saliva, tears, bile) that contains cells or cell components.

The disclosed systems and methods further comprise evaluating white matter microstructure and the hemispheric organization of white matter for one or more animal models which include, but are not limited to, animal models of neurological disorders or diseases and animal models involving mismyelination, demyelination, or dysmyelination. The animal model can be a transgenic animal expressing a nucleic acid encoding a polypeptide or peptide that has, or can have, a role in the development or propagation of a neurological disease or disorder. For example, such a polypeptide or peptide can affect myelination. The nucleic acid encoding a polypeptide or peptide can have, or be suspected to have, a role in the development or propagation of a neurological disease or disorder.

Also disclosed herein are systems and methods of identifying the presence or absence of a neurological disease or disorder by evaluating white matter microstructure and the hemispheric organization of white matter comprising a transgenic animal. A transgenic animal can be produced by the process of transfecting a cell within the animal with any of the nucleic acid molecules disclosed herein. The art is familiar with methods for producing transgenic animals (see, e.g., U.S. Pat. No. 6,201,165, which is hereby incorporated in its entirety by reference). The transgenic animal can be a mammal, such as a mouse, rat, rabbit, cow, sheep, pig, or primate, such as a human, monkey, ape, chimpanzee, or orangutan. The transgenic animal also can be an animal produced by the process of adding to such animal (for example, during an embryonic state) any of the cells disclosed herein.

The art is familiar with the compositions (such as vectors) and methods that can be used for targeted gene disruption and modification to produce polypeptides of interest in any animal that can undergo gene disruption. Polypeptides of interest include but are not limited to polypetides that have, or can have, a role in the development or propagation of a neurological disease or disorder. For example, such a polypeptide or peptide can affect myelination. The nucleic acid encoding a polypeptide or peptide also can have, or be suspected to have, a role in the development or propagation of a neurological disease or disorder.

G7ene modification and gene disruption refer to the methods, techniques, and compositions that surround the selective removal or alteration of a gene or stretch of chromosome in an animal, such as a mammal, in a way that propagates the modification through the germ line of the mammal. Generally, a cell is transformed with a vector, which is designed to homologously recombine with a region of a particular chromosome contained within the cell, as for example, described herein. This homologous recombination event can produce a chromosome which has exogenous deoxyribonucleic acid (DNA) introduced, for example in frame, with the surrounding DNA. This type of protocol allows for very specific mutations, such as point mutations or the insertion of DNA to encode for a new polypeptide, to be introduced into the genome contained within the cell. Methods for performing this type of homologous recombination are known to one of skill in the art.

After a genetically engineered cell is produced through the methods described above, an animal can be produced from this cell through either stem cell technology or cloning technology. For example, if the cell into which the nucleic acid was transfected was a stem cell for the organism, then this cell, after transfection and culturing, can be used to produce a transgenic organism which will contain the gene modification or disruption in germ line cells, which can then in turn be used to produce another animal that possesses the gene modification or disruption in all of its cells. In other methods for production of an animal containing the gene modification or disruption in all of its cells, cloning technologies can be used. These technologies are known to one of skill in the art and generally take the nucleus of the transfected cell and either through fusion or replacement fuse the transfected nucleus with an oocyte, which can then be manipulated to produce an animal. The advantage of procedures that use cloning instead of ES technology is that cells other than ES cells can be transfected. For example, a fibroblast cell, which is very easy to culture and can be used as the cell in this example, which is transfected and has a gene modification or disruption event take place, and then cells derived from this cell can be used to done a whole animal. After the transgenic animal is created, the systems and methods disclosed herein can be used to identify the presence or absence of a neurological disease or disorder in the transgenic animal. The systems and methods disclosed herein also can be used to evaluate white matter microstructure or the hemispheric organization of white matter or both in the transgenic animal.

In an aspect, embodiments of the subject disclosure can be employed to evaluate the progression of a subject's neurological disease or neurological disorder. A clinician (e.g., a medical doctor) or researcher can utilize the embodiments of the subject disclosure to acquire data regarding the subject's white matter microstructure or white matter hemispheric organization at scheduled times. For example, in certain embodiments in which a subject is human, the acquisition of data can be performed periodically, wherein the scheduled times occur at regular intervals, such as every 3 months, 6 months, 9 months, or every year, every other year, every 5 years, every 10 years for the life of the subject. In alternative or additional embodiments, the scheduled times need not be periodic. For another example, in embodiments in which the subject is non-human, the acquisition of data can be carried out periodically at scheduled times spaced at regular intervals, such as every week, every other week, every month, every other month, every 3 months, every 6 months, every 9 months, every year, every other year for the life of the non-human subject. In an aspect, in view of physiologic differences between humans and non-humans, which can dictate disparate progression of a neurological disorder or neurological disease, imaging data directed to probing a CNS structure in a non-human subject can be observed in time scales that are different than those in a human subject.

Using the disclosed systems and methods to evaluate the progression of a subject's neurological disease or neurological disorder can further comprise evaluating the effect of various treatments on white matter microstructure and the hemispheric organization of white matter. For example, the ability of various treatments to modulate white matter microstructure and the hemispheric organization of white matter can be evaluated. As used herein, the term “modulate” is meant to alter, by increasing or decreasing. Modulate can refer to an alteration of white matter microstructure, for example, so that the white matter microstructure and the hemispheric organization of white matter is more or less asymmetric or more or less atypical when compared to that of a normal, non-affected subject. Modulate also can refer to an alteration in the biological activity of a gene or peptide, which, in turn, can affect white matter microstructure and the hemispheric organization of white matter, for example, through mismyelination, demyelination, or dysmyelination. Modulation may be an increase or a decrease in peptide activity, a change in binding characteristics, or any other change in the biological, functional, or immunological properties of the peptide.

Treatment as used herein can refer to various types of compositions, techniques, therapies, and devices, that can be used to affect a neurological disease or disorder, or affect the white matter microstructure or white matter hemispheric organization associated with a neurological disease or disorder. For example, treatment can comprise a chemical, a pharmaceutical agent, or combinations thereof, which can be administered to a subject to treat a neurological disease or disorder. Treatment can comprise surgical intervention. Treatment can comprise therapy, which is directed at the subject's emotional, cognitive, vocal, social, and physical skills Treatments can be delivered or exercised alone or can be delivered or exercised in combination with one or more other forms of treatment. Treatment can be repeatedly or continuously delivered. Such treatment can affect the subject's susceptibility for developing a neurological disease or disorder, in order to prevent or delay a worsening of the effects of the disease or disorder, or to partially or fully reverse the effects of the disease or disorder, including the underlying white matter microstructure.

To “treat” also can refer to non-pharmacological methods of preventing or delaying a worsening of the effects of a neurological disease or disorder, including the underlying white matter microstructure and white matter hemispheric organization, or to partially or fully reversing the effects of the neurological disease or disorder. For example, “treat” is meant to mean a course of action to prevent or delay a worsening of the effects of the disease or disorder or to partially or fully reverse the effects of the neurological disease or disorder other than by administering a compound.

A clinician or researcher can utilize the disclosed systems and methods to evaluate the effects of treatment on a neurological disease or disorder, for example, by acquiring data regarding the subject's white matter microstructure and white matter hemispheric organization following treatment or in conjunction with treatment. For example, in a human subject, as treatment can be repeatedly or continuously delivered, the acquisition of data can be repeated at regular intervals such as every 3 months, 6 months, 9 months, or every year, every other year, every 5 years, or every 10 years for the life of the subject. For a non-human subject, as treatment can be repeatedly or continuously delivered, the acquisition of data can be repeated, for example, every week, or every other week, or every month, or every other month, or every 3 months, or every 6 months, or every 9 months, or every year, or every other year for the life of the non-human subject. The subject disclosure and the various embodiments thereof described herein also can be employed to evaluable the progression of a subject's neurological disease or disorder following the administration of treatment or in conjunction with the administration of the treatment.

The subject disclosure and the various embodiments thereof described herein also can distinguish one neurological disorder or disease from another. For example, using the disclosed systems and methods can generate a representative or composite map for normal, or control, white matter microstructure and the hemispheric organization of white matter. Using the disclosed embodiments, a representative or composite value for multiple tensor metrics can be generated. Such a composite map can be constructed, for example, by imaging multiple normal subjects. Similarly, such a composite tensor metric can be generated by collecting DTI-MRI data from multiple normal subjects. By comparing the control, or normal, white matter microstructure and hemispheric organization of white matter to the white matter microstructure and hemispheric organization of white matter of a subject suspected to have, or believed to have, a neurological disease or neurological disorder, a clinician or researcher can identify white matter asymmetries or atypicalities, which in turn, can allow for a diagnosis of the exact neurological disease or neurological disorder, or a likely candidate neurological disease or neurological disorder.

Classification research in autism seeks to discover objective in vivo biological measurements that distinguish individuals with autism from typically developing individuals and those with other developmental and neuropsychiatric disorders. To have clinical value as a biomarker for the disorder, these measurements should demonstrate very high sensitivity, specificity, accuracy, reliability and predictive value. To date, the proposed metrics show inadequate classification ability, or classification performance, and have not been replicated validated or replicated in an independent sample.

Relationship of brain asymmetry to language has been studied for over a century. The possibility that autism and other language disorders of childhood can be due to atypical asymmetric brain development has intrigued clinicians and researchers for over a quarter of a century. As in other developmental language disorders, populations with autism generally exhibit less right-handedness, atypical brain asymmetries, and altered callosal neuroanatomy and function. However, the in vivo neuropathology of autism and its relationship to language dysfunction remain unclear. The subject disclosure and the various embodiments thereof described herein can quantify hemispheric asymmetry and fiber organization of white matter microstructure (WMM) in the superior temporal gyrus and temporal stem in autism. Based at least on such quantification, the subject disclosure enables investigation of a possible link to language dysfunction that is typical of the disorder. More generally, for a neurological disease or neurological disorder that afflict a central nervous system (CNS) structure, the subject disclosure enables quantification of spatial asymmetry amongst portions of the CNS structure, and organization of WMM therein.

Conventional brain development studies in autism have found associations between functional hemispheric asymmetry deviations and language functioning and cognitive ability impairments in the absence of volumetric differences. The pathogenesis of autism can involve atypical inter-hemispheric organization of white matter microstructure. The superior temporal gyrus (STG) and temporal stem (TS) are important in autism due to their roles in language, emotion, and social cognition. The STG exchanges information with the rest of the brain via afferent and efferent pathways with the TS and, through the arcuate fasciculi, to the inferior frontal gyrus (direct long segment) via Geschwind's area in the inferior parietal lobule (indirect short segment). The WMM of the STG and TS is as important as that of other brain regions affected in autism.

In addition, conventional studies have shown that greater volumetric white matter differences in autism exist in superficial versus deep white matter compartments, and that regional temporal lobe volume deviations exist in both compartments. Although volumetric STG asymmetry has not been found, functionally asymmetric STG differences have been observed repeatedly in autism. The TS contains functionally diverse, bidirectional fibers subserving multiple aspects of language and social cognition including memory, facial recognition, emotional processing, and reactivity, which are abilities usually impaired in autism. Since WMM asymmetry appears central to typical functional lateralization, its atypicality in autism could underlie functionally asymmetric aberrations in the absence of changes in regional white matter volumes.

Moreover certain earlier, conventional studies of STG white matter microstructure found evidence of its pathology in autism, including clusters of FA reduction and differences in FA and MD likely due to increased D_{R}. Age-related deviations in WMM during childhood, adolescence, and young adulthood can be specific to autism. Fractional anisotropy has been observed to increase in young autistic children and decrease in older children, adolescents, and adults with the disorder. Age-dependent group differences have been found only in STG FA, corpus callosum, and the posterior limb of the right internal capsule. Although functionally asymmetric age-related changes in auditory cortex have been reported in autism, age effects on regional WMM asymmetry have not been examined. Through the use of a new tensor shape metric, tensor skewness, such studies analyzed STG and TS white matter microstructure asymmetry in subjects meeting full diagnostic criteria for autism—not only for the broader autism spectrum disorders—who were tightly matched to a control.

The subject disclosure and the various embodiments thereof described herein allow to address several aspects or features concerning STG and TS white matter microstructure in autism relative to typical development. For example, the subject disclosure addressed (1) whether atypical hemispheric asymmetry is present; (2) whether dosage-related asymmetry deviations exist; (3) whether differences in tensor skewness and asymmetry thereof exist; (4) whether asymmetry can be affected by psychiatric comorbidity and medication status; (5) whether language impairments can be related to asymmetry differences amongst autism and typical development; and (6) whether a limited small number of STG and TS white matter microstructure metrics can discriminate between subjects with autism and typically developing subjects.

### I. Example Embodiments

### A. Participants

Thirty high-functioning (performance IQ (PIQ)≧85) right-handed males, who all met full criteria for autism, were matched with thirty (30) typically developing males on age, PIQ, handedness, and head circumference. All participants, or subjects, were scanned during a two-year period as part of an ongoing longitudinal autism study. All participants provided written informed consent or assent prior to participation.

### B. Diagnosis

Autism diagnosis was based on ADI-R, ADOS-G, DSM-IV, and ICD-10 criteria as known to a person having ordinary skill in the art. Exclusion criteria included patient history, Fragile-X, karyotype or clinical indications of medical causes of autism, history of severe head injury, hypoxia-ischemia, seizures, and other neurologic disorders. Psychiatric comorbidity was not an exclusion criterion since it occurs in the majority of children and adults with autism. Lifetime psychiatric comorbidity was identified in 53% (16/30) of the subjects. Of these sixteen subjects, 56% (9 subjects) had depression, 31% (5 subjects) had attention deficit/attention deficit hyperactivity disorder, 25% (4 subjects) had obsessive-compulsive disorder, 19% (3 subjects) had anxiety disorder. In an aspect, at the time of testing, sixty-three percent (19/30) of subjects with autism were taking one or more psychotropic medications. Of these nineteen subjects, 89% (17 subjects) used SSRIs, 26% (5 subjects) used stimulants, 26% (5 subjects) used valproic acid, and 26% (5 subjects) used neuroleptics. Control participants, or control subjects, were tested for IQ and language function by standardized assessments and the ADOS-G to confirm typical development. In another aspect, control subjects (also referred to as controls) with any evidence of developmental, learning, cognitive, neurological, or neuropsychiatric conditions were excluded.

### C. Assessments

In the subject disclosure, the following clinical assessments were performed: (i) Handedness—assessed using the Edinburgh Handedness Inventory. (ii) Head circumference—measured as maximal occipital-frontal head circumference. (iii) IQ—verbal IQ and PIQ were ascertained with the DAS or WISC-III for children and the WAIS-III for adults. (iv) Language—at the time of testing, all participants were verbal and spoke English as their first language. The CELF-3 measured language functioning. Word Selective Reminding (WSR), a subtest of the TOMAL, quantified immediate verbal recall. (v) Quantitative Measure of Autistic Traits—the Social Responsiveness Scale (SRS) quantified autism phenotype. (vi) Psychiatric comorbidity—the Autism Comorbidity Interview (ACI) assessed lifetime history of comorbidity and ruled out any concurrent episode of major depression.

### D. Diffusion Tensor Magnetic Resonance Imaging (DT-MRI)

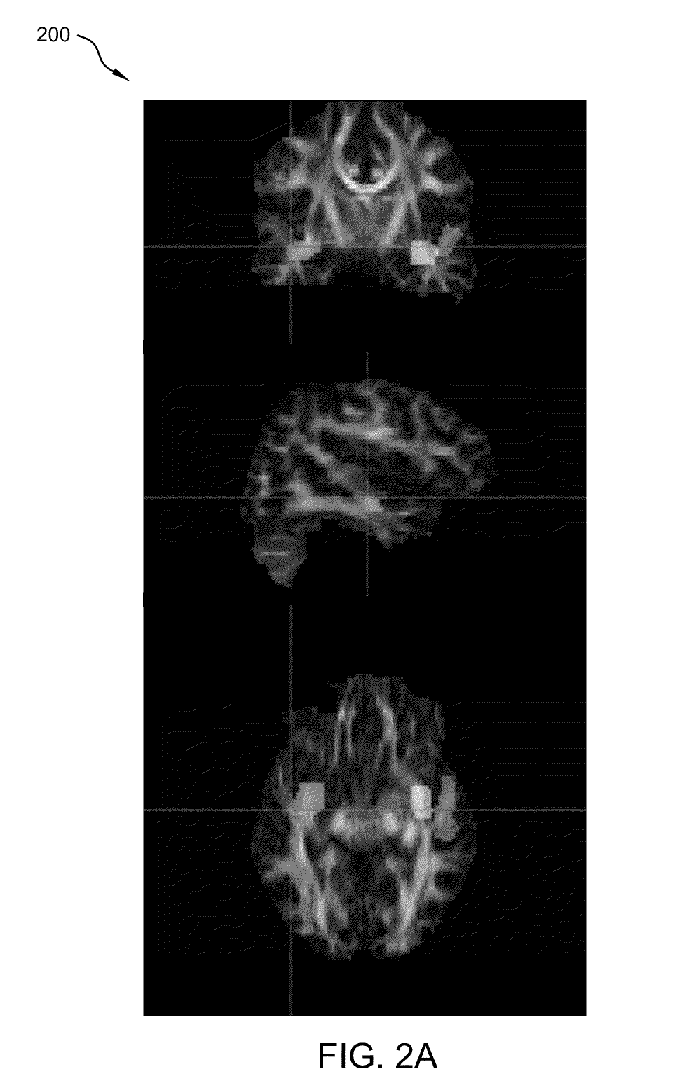

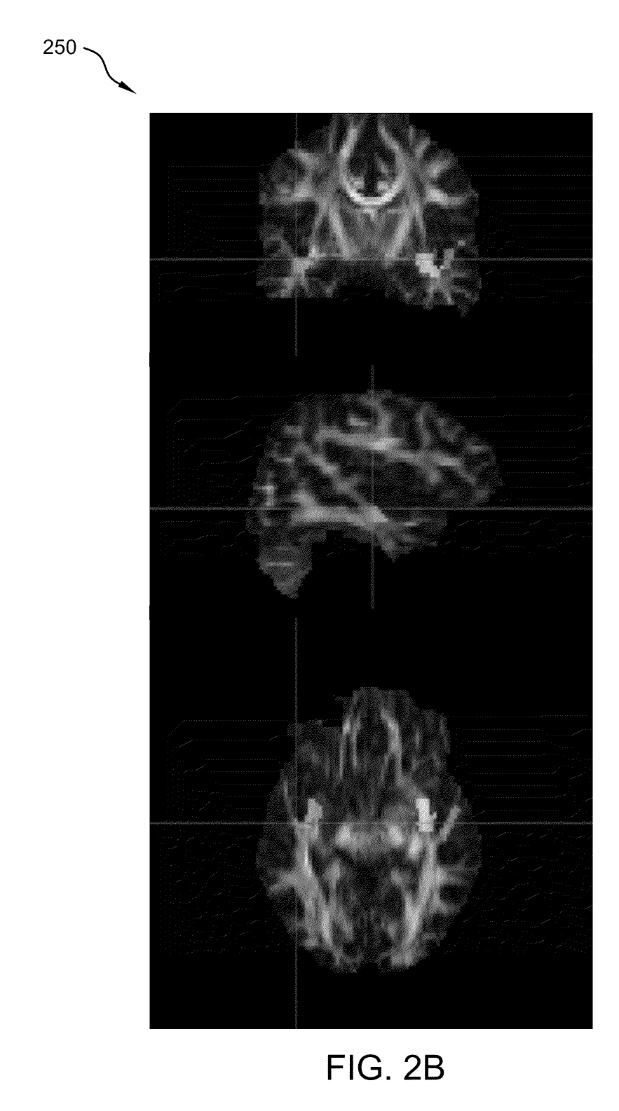

Brain imaging was performed on a Siemens Trio 3.0 Tesla scanner; in certain scenarios, such scanner embodies at least in part the imaging unit 110. In an aspect, an anesthesiologist sedated three children with autism and monitored them continuously following American Society of Anesthesiology standards. There were no complications. Echo-planar imaging (EPI) with parallel imaging (SENSE factor 2) was employed, as was dual-refocused bipolar diffusion-weighting gradients, an 8-channel receiver coil to acquire a b=0 reference volume, and 12 diffusion-weighted volumes with non-collinear encoding directions in 50 contiguous, 2.5 mm thick axial slices (in-plane resolution=2 mm; field of view (FOV)=256 mm; matrix=128×128; TR/TE=7000/84 ms; pixel bandwidth=1345 Hz; 4 averages). In certain scenarions, diffusion-weighting gradients were applied at rate of about b=1000 s/mm² in each direction probed in the brain or other nervous tissue. A field map was generated from a 2D gradient-echo image pair (TE1=4.32 ms, TE2=6.78 ms). Corrections for eddy currents, head movements, and EPI distortions were performed. The corrected images were interpolated to 2 mm isotropic voxels. Volumes of STG white matter and TS white matter were extracted using the subject images registered to a regional template. Diffusion tensor eigenvalues, MD, FA, D_{A} and D_{R} also were calculated. FIG. 2A is a diagram 100 of example segmentation of the STG and TS in diffusion tensor magnetic resonance images for raw imaging data, whereas FIG. 2B is a diagram 150 for imaging data masked for white matter only.

In an aspect, for the group discrimination and classification study, employing DT-MRI measures only, tensor skewness was examined. Tensor skewness also is commonly known as tensor mode. Tensor skewness is a measure of a distinct component of directional diffusion coherence that is separable from and additional to that measured by FA only. Geometrically, tensor skewness quantifies the degree of prolate tensor shape, e.g., increased directional diffusion coherence (or “linear anisotropy”) versus oblate tensor shape, or more directionally incoherent diffusions (or “planar anisotropy”), and is mathematically orthogonal to both FA and the root mean square of the tensor eigenvalues.

### E. Statistical Analysis

Diffusion tensor metrics for a tensor diffusion field are summarized by an associated non-empty set of coefficients: hemispheric mean, standard deviation (SD), and coefficient of variation (CV). The coefficient of variation can be defined as the SD expressed as a fraction of the mean (e.g., CV=SD/mean). In an aspect, CV is preferred to SD in scenarios in which comparing mean-variance pairs that can differ in aggregate and also can be correlated. For each tensor metric, repeated-measures ANCOVA accounted for associations of a tensor metric with a diagnosis of autism, hemisphere, age, total brain volume (TBV), handedness, PIQ, and their inter-hemispheric correlations. In another aspect, a hemispheric asymmetry index can be defined as η=2(L−R)/(L+R), wherein positive and negative values of η indicate, respectively, leftward and rightward asymmetry. It should be appreciated that alternative or additional definitions of hemispheric asymmetry can be effected and utilized. In the subject disclosure, test-wise false-positive error rate is set at 0.05. Substantially all or all P-values were corrected by Bonferroni's method (factor of 4). In yet another aspect, the effects of white matter microstructure hemispheric asymmetry on language functioning and social cognition are examined while covarying for comorbidity and medication usage. Computing unit 120 or one or more functional elements therein (unit(s), component(s), platform(s), etc.) can generate the non-empty set of coefficients indicated hereinbefore. Moreover, computing unit 120 can supply at least one element of such non-empty set to data storage 130 to retain a record of the at least one element.

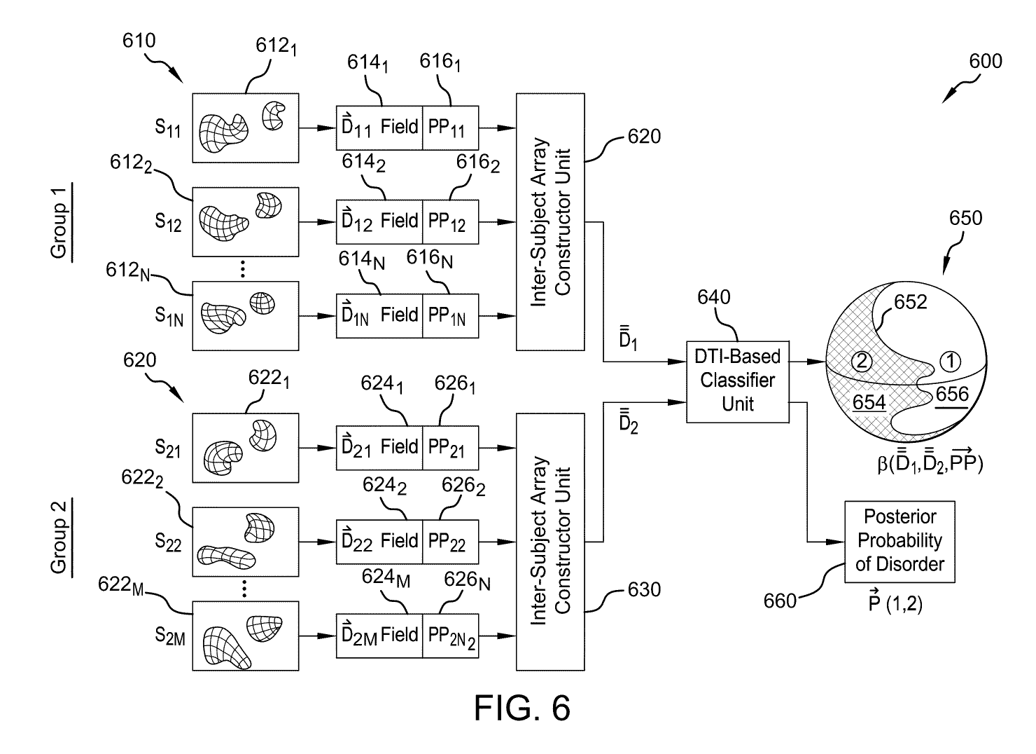

In an embodiment, quadratic discriminant analysis (QDA) with leave-one-out cross-validation and computation of Mahalanobis distances was implemented to determine the combination of tensor metrics that minimized group misclassification rate, favoring sensitivity over specificity. In another embodiment, a support vector machine (SVM) with Gaussian kernel and leave-one-out cross-validation also was utilized to compare parametric and non-parametric approaches. FIG. 6 is a block diagram of an example system 600 that enables generation of a multivariate classifier that can supply a likelihood, or probability, of a subject having a neurological disease or neurological disorder. In example system 600, imaging data for a first group of N subjects (N a positive integer) and a second group of M (M a positive integer) subjects is generated; for instance, imaging unit 110 can produce the imaging data in accordance with aspects described hereinbefore. For the first group, the imaging data is generated for each subject S_{1G} in the group, with G=1, 2 . . . N. For a subject G (with 1≦G≦N), imaging data 612_{G} correspond to diffusion tensor field 614_{G}; such field is formatted as a vector \vec{D}_{1G} and is assigned a prior probability (PP) 616_{G} of subject S_{1G} having the neurological disease or neurological disorder. Similarly, for the second group, the imaging data is generated for each subject S_{2H} in the group, with H=1, 2 . . . M. For a subject H (with 1≦H≦M), imaging data 622_{H} correspond to diffusion tensor field 624_{H}; such field is formatted as a vector \vec{D}_{1H} and is assigned a prior probability (PP) 616_{G} of subject S_{1G} having the neurological disease or neurological disorder. Prior probabilities PP 616_{G} and PP 626_{H} can be assigned, respectively, values PP_{1G} and PP_{1H} which can be equal or substantially equal to 0.5. In an aspect, PP 616_{1G} and PP 626_{H} can be defined based at least in part on at least one clinical metric, such as one of the metrics employed in conventional clinical assessment of the neurological disease (e.g., autism) or neurological disorder. Inter-subject array constructor unit 620 (also referred to as inter-subject array constructor 620) receives the set of N composite quantities (\vec{D}_{1G}; PP_{1G}) with G=1, 2 . . . N, and generates a T-tuple D₁ with T=6 VN, wherein V is the number of voxels that define the dimension of the diffusion tensor field. Likewise, inter-subject array constructor unit 630 (also referred to as inter-subject array constructor 630) receives the set of M composite quantities (\vec{D}_{1H}; PP_{1H}) with H=1, 2 . . . M, and generates a T-tuple D₂ with T=6 V′M, wherein V′ is the number of voxels that define the dimension of the diffusion tensor field. In the illustrated example embodiment, inter-subject array constructor unit 620 and inter-subject array constructor unit 630 supply, respectively, D₁ and D₂ to DTI-based classifier unit 640.

DTI-based classifier unit 640 computes a set of values of a group of diffusion tensor metrics, wherein the rank R of such group defines an R-dimensional sub-space of classification features. Based at least on the set of values and a statistical machine-learning model, DTI classifier unit 640 generates a multivariate classifier that distinguishes amongst presence or absence of the neurological disease. In certain embodiments, the machine-learning model is discriminant analysis, e.g., QDA. In such embodiments, the multivariate classifier can be represented by a discrimation function that defines a decision boundary in the R-dimensional sub-space of classification features. In the depicted example system 600, the a hypersphere 650 represents the R-dimensional sub-space of classification features and a hyperplane 652 represents the decision boundary, which distinguishes between a first portion 656 and a second portion 654 of the hypersphere 650. In addition, DTI-based classifier unit 640 can supply (e.g., compute and deliver) a posterior probability of disorder 660—in example system 660, for the first group and the second group, DTI-based classifier unit 640 supplies a vector indicative of such probability. Thus, DTI-based classifier unit 640 can provide a likelihood of presence or absence of the neurological disease or disorder and, therefore, enables identification of such presence or absence. In alternative or additional embodiments, the statistical machine-learning model is a support vector machine. In another alternative or additional embodiment, the statistical learning model is a neural network.

It should be appreciated that to carry out the statistical analysis, the DTI-based classifier unit 640 implements the statistical machine-learning model, e.g., DTI-based classifier unit 640 executes at least one computer-executable instruction representative of such model. The at least one computer-executable instruction can be part of one or more non-empty sets of computer-executable instructions. In an aspect, statistical analysis can be implemented through execution of a commercial or open-source software or firmware application, which can embody the one or more non-empty sets of computer-executable instructions. In an example implementation, quadratic discriminant analysis of imaging data is performed in R version 2.9.0 (04/17/09 build) which yields results equivalent to those produced by SAS. In an example implementation, the support vector machine was computed with a custom-designed software package (LIBSVM software package available from http://www.csie.ntu.edu.tw/˜cjlin/libsvm). The one or more non-empty sets of computer-executable instructions can be retained in data storage 130 or in computing unit 120.

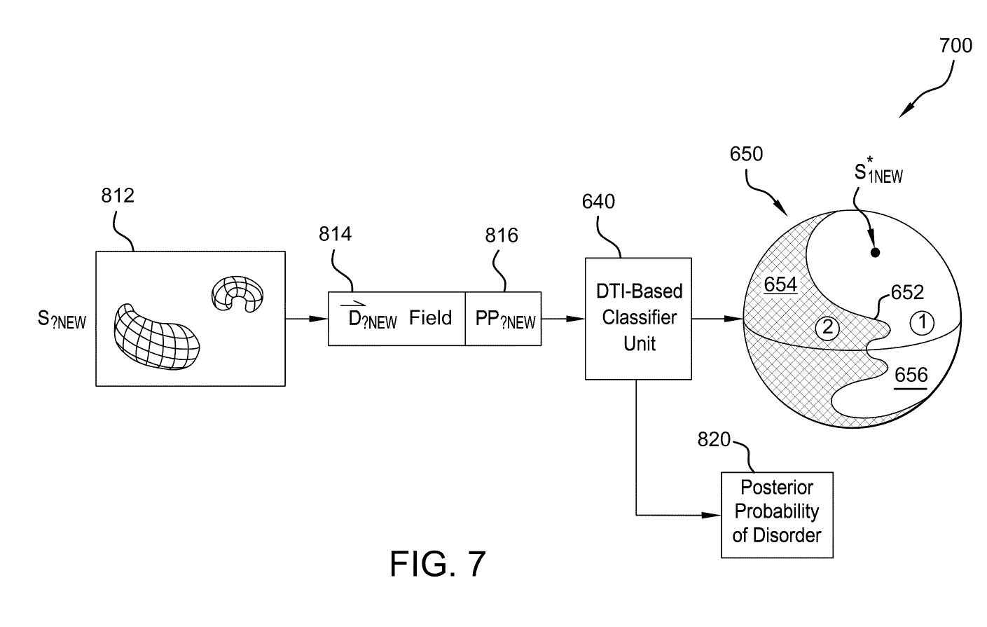

In an aspect, classification ability of the multivariate classifier was determined by an independent 30% replication group of test subjects not employed in the QDA: For autism, the replication group included 12 subjects, and for control, the replication group included seven (7) typically developing subjects. Reliability of the classification algorithm was assessed by the intraclass correlation coefficient, equivalent to Cohen's κ. As an illustration of assessment of classification ability, FIG. 7 is a diagram 700 that depicts implementation of the multivariate classifier to a test subject S_{?New}, wherein the label “?New” indicates that the subject is a test subject and has not been part of the machine-learning stage associated with generation of the multivariate classifier. It should be appreciated that formatting of imaging data need not be performed as described hereinbefore. Instead, in implementation of such multivariate classifier, imaging data 814 (\vec{D}_{?New}) for the test subject is linked to a prior probability PP_{?New} of having the neurological disease or neurological disorder, and the composite data structure is supplied to DTI-based classifier unit 640. In response, DTI-based unit applies the multivariate classifier to a set of values of the group of diffusion tensor metrics that span the sub-space of classification features considered for generation of the multivariate classifier, wherein DTI-based classifier unit 640 extracts such set of values from the composite data structure (\vec{D}_{?New};PP_{?New}). In response to application of the multivariate classifier, DTI-based classifier unit 640 classifies the test subject as having the neurological disease or not having it, and supplies a likelihood of presence of the neurological disease; such likelihood is embodied in the posterior probability of disorder 820, which is specific to the test subject. In the illustrated example, the test subject S_{?New} is classified as having the neurological disease.

In an aspect, statistical analysis also reveals atypical loss and reversal of leftward asymmetry. As another example, statistical analysis also reveals atypical reductions in spatial organization of white matter fibers and atypical age-related decreases of white matter microstructure in the superior temporal gyrus and temporal stem in autism. A multivariate classifier based in part on a feature sub-space including six of these metrics and generated from the learning samples discriminated between control and autism subjects with 91.6% accuracy, 93.6% sensitivity, 89.6% specificity, 90% positive predictive value, 93.3% negative predictive value, and 83.3% reliability. Application of the multivariate classifier to the independent group yields an increased discrimination accuracy of 94.7%.

### II. Results

### A. Participant Characteristics

No significant differences between the autism and control groups with respect to age, IQ, handedness, or head circumference were found (see, e.g., Table 1). Groups differed in tests of language ability and social responsiveness. There was no evidence of greater subject motion in the autism sample. The following notation applies to Table 1: Symbol * indicates that the results were not statistically significant at a false-positive error rate 0.05 and a P-value greater than 0.20; symbol ^{†} indicates that n=27 for control subjects and n=28 for autism subjects; ^{‡} indicates the Edinburgh Handedness Inventory where the range extends from −100 (left handed) to 100 (right handed); symbol ^{§} indicates the Clinical Evaluation of Language Fundamentals 3 (CELF-3), where n=28 for control subjects and n=30 for autism subjects; and symbol ** indicates Responsiveness Scale (RS), for a child, or Social Reciprocity Scale (SRS), for an adult, where the range extends from 0 to 195 for control subjects (n=27) and autism subjects (n=28), with a value of 0 indicating absence of autistic-like traits and a value of 195 indicating several severe autistic traits. Here, the verification of the 7 subjects having SRS scores less than 85 (SRS score 34, 1; 62-72, 2; 76-84, 4) confirmed that the subjects met full diagnostic criteria for autism.

TABLE 1
Physical and Cognitive Ability Characteristics of the Samples

| | | | | | Between-Group | |
|---|---|---|---|---|---|---|
| | Control (n = 30) | | Autism (n = 30) | | Comparison | |
| | Mean (SD) | Range | Mean (SD) | Range | t value | P value |
| Age (years) | 15.79 (5.5) | 8.1-26.3 | 15.78 (5.6) | 7.0-27.8 | 0.10 | n.s.* |
| Head | 56.00 (2.1) | 52-59 | 56.63 (2.3) | 53-60 | 1.13 | n.s. |
| Circumference^{†} | | | | | | |
| Handedness^{‡} | 75.17 (24.9) | 6-100 | 80.07 (22.6) | 13-100 | 0.48 | n.s. |

Intelligence Quotient

| | | | | | Between-Group | |
|---|---|---|---|---|---|---|
| | Control (n = 30) | | Autism (n = 30) | | Comparison | |
| | Mean (SD) | Range | Mean (SD) | Range | t value | P value |
| Full-scale IQ | 115.13 (12.9) | 94-135 | 109.57 (16.7) | 80-140 | 1.40 | n.s. |
| Performance IQ | 112.77 (12.5) | 90-134 | 109.43 (13.5) | 85-135 | 0.88 | n.s. |
| Verbal IQ | 112.80 (13.2) | 90-140 | 106.63 (21.6) | 70-145 | 1.34 | n.s. |

Language Functioning^{§}

| | | | | | Between-Group | |
|---|---|---|---|---|---|---|
| | Control (n = 30) | | Autism (n = 30) | | Comparison | |
| | Mean (SD) | Range | Mean (SD) | Range | t value | P value |
| Total | 109.5 (13.2) | 84-137 | 91.34 (21.3) | 50-123 | 3.85 | <0.001 |
| Receptive | 110.0 (15.9) | 82-143 | 93.85 (24.7) | 50-125 | 2.76 | 0.008 |
| Expressive | 106.9 (12.2) | 82-131 | 90.22 (20.0) | 50-120 | 3.58 | 0.001 |

| | | | | | Between-Group | |
|---|---|---|---|---|---|---|
| | Control (n = 30) | | Autism (n = 30) | | Comparison | |
| | Mean (SD) | Range | Mean (SD) | Range | t value | P value |
| SRS** | 15.9 (13.1) | 0-48 | 99.61 (24.0) | 34-148 | 15.93 | <0.001 |

### B. Tensor Metrics by Group, Structure, and Hemisphere

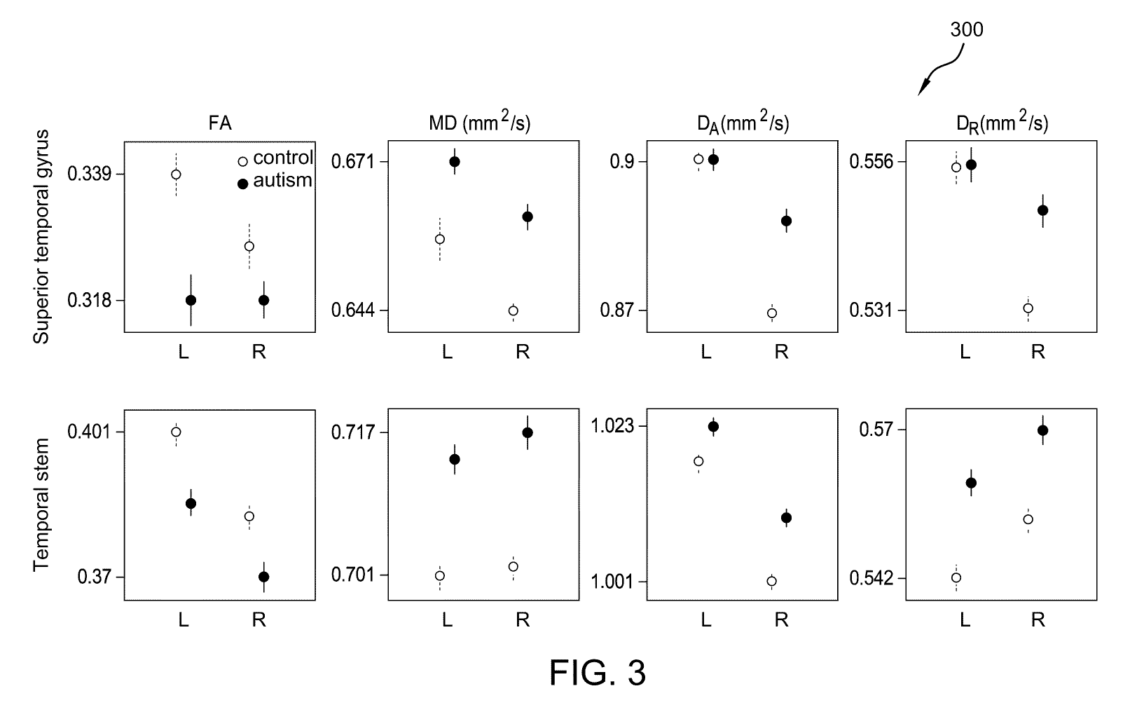

Tables 2A-2C contains the mean, SD, and CV of left (L) and right (R) STG and TS tensor metrics and asymmetry indices. FIG. 3 is a diagram 300 that pictorially represents values in Table 2A. The following notation applies to results in Tables 2A-2C: Symbol * indicates η=2(L−R)/(L+R); symbol ^{†} indicates CV=SD/mean; and symbol ^{‡} indicates that data could not be interpreted due to a near singularity, or near-zero denominator. In terms of CV, a realization of a result having CV<0.01 is uninterpretable.

TABLE 2A
Tensor Metrics and Asymmetry Indices by Group and Hemisphere

| Control (n = 30) | | | | | | | | |
|---|---|---|---|---|---|---|---|---|
| Superior | | | | | Autism (n = 30) | | | |
| Temporal Gyrus | | | | Asymmetry | | | | Asymmetry |
| (STG) | | Left | Right | Index* | STG | Left | Right | Index |
| FA | Mean | 0.339 | 0.327 | 0.0373 | Mean | 0.318 | 0.318 | −0.0024 |
| | (SD) | (0.020) | (0.024) | (0.0529) | (SD) | (0.024) | (0.018) | (0.0582) |
| | CV^{†} | 0.059 | 0.073 | 1.4182 | CV^{†} | 0.075 | 0.057 | Ø^{‡} |
| MD | Mean | 0.657 | 0.644 | 0.0194 | Mean | 0.671 | 0.661 | 0.0142 |
| (mm²/s) | (SD) | (0.027) | (0.020) | (0.0253) | (SD) | (0.027) | (0.029) | (0.0162) |
| | CV^{†} | 0.041 | 0.031 | 1.3041 | CV^{†} | 0.040 | 0.044 | 1.1408 |
| D_{A} | Mean | 0.900 | 0.870 | 0.0336 | Mean | 0.900 | 0.888 | 0.0132 |
| (mm²/s) | (SD) | (0.035) | (0.025) | (0.0380) | (SD) | (0.031) | (0.035) | (0.0337) |
| | CV^{†} | 0.039 | 0.029 | 1.1310 | CV^{†} | 0.034 | 0.039 | 2.5530 |
| D_{R} | Mean | 0.535 | 0.531 | 0.0074 | Mean | 0.556 | 0.548 | 0.01500 |
| (mm²/s) | (SD) | (0.027) | (0.023) | (0.0232) | (SD) | (0.030) | (0.029) | (0.0192) |
| | CV^{†} | 0.050 | 0.043 | 3.1351 | CV^{†} | 0.054 | 0.053 | 1.2800 |

| | | Control (n = 30) | | | Autism (n = 30) | | | |
|---|---|---|---|---|---|---|---|---|
| Temporal Stem | | | | Asymmetry | | | | Asymmetry |
| (TS) | | Left | Right | Index* | TS | Left | Right | Index |
| FA | Mean | 0.401 | 0.383 | 0.0463 | Mean | 0.386 | 0.370 | 0.0439 |
| | (SD) | (0.021) | (0.019) | (0.0384) | (SD) | (0.019) | (0.022) | (0.0440) |
| | CV^{†} | 0.052 | 0.050 | 0.8294 | CV^{†} | 0.049 | 0.059 | 1.0023 |
| MD | Mean | 0.701 | 0.702 | −0.0028 | Mean | 0.714 | 0.717 | −0.0037 |
| (mm²/s) | (SD) | (0.020) | (0.019) | (0.0177) | (SD) | (0.020) | (0.023) | (0.0162) |
| | CV^{†} | 0.029 | 0.027 | Ø^{‡} | CV^{†} | 0.028 | 0.032 | Ø^{‡} |
| D_{A} | Mean | 1.018 | 1.001 | 0.0164 | Mean | 1.023 | 1.010 | 0.0128 |
| (mm²/s) | (SD) | (0.028) | (0.020) | (0.0225) | (SD) | (0.021) | (0.022) | (0.0143) |
| | CV^{†} | 0.028 | 0.020 | 1.3720 | CV^{†} | 0.021 | 0.022 | 1.1172 |
| D_{R} | Mean | 0.542 | 0.553 | −0.0205 | Mean | 0.560 | 0.570 | −0.0184 |
| (mm²/s) | (SD) | (0.022) | (0.023) | (0.0252) | (SD) | (0.023) | (0.027) | (0.0272) |
| | CV^{†} | 0.041 | 0.042 | −1.2293 | CV^{†} | 0.041 | 0.047 | −1.4783 |

TABLE 2B
Tensor Coefficients and Asymmetry
Indices by Group and Hemisphere

| Typically developing (TD) | | | | | |
|---|---|---|---|---|---|
| N = 30 | | | Autism N = 30 | | |
| Left | Right | AI | Left | Right | AI |
| Mean | Mean | Mean | Mean | Mean | Mean |
| (SD) | (SD) | (SD) | (SD) | (SD) | (SD) |
| CV | CV | CV | CV | CV | CV |

Superior Temporal Gyrus

| | | | | | | |
|---|---|---|---|---|---|---|
| Skewness | 0.517 | 0.502 | 0.0303 | 0.505 | 0.517 | −0.022 |
| | (0.049) | (0.052) | (0.0969) | (0.04) | (0.049) | (0.1009) |
| | 0.095 | 0.103 | 3.1956 | 0.078 | 0.095 | 4.5838 |

Temporal Stem

| | | | | | | |
|---|---|---|---|---|---|---|
| Skewness | 0.622 | 0.627 | 0.008 | 0.62 | 0.611 | 0.0142 |
| | (0.032) | (0.035) | (0.0644) | (0.035) | (0.032) | (0.0628) |
| | 0.051 | 0.057 | 8.0024 | 0.057 | 0.052 | 4.4075 |

TABLE 2C
Coefficients and Asymmetry Indices by Group and Hemisphere:
Inter-group Autism-TD Comparison

| | Left | Right | AI |
|---|---|---|---|
| | Mean | Mean | Mean |
| | (SD) | (SD) | (SD) |
| Superior | | | |
| Temporal | | | |
| Gyrus | | | |
| Skewness | −0.012 | 0.015 | −0.0523 |
| | (0.063) | (0.071) | (0.1399) |
| FA | −0.021 | −0.009 | −0.0397 |
| | (0.031) | (0.03) | −0.0786 |
| MD (mm²/s) | 0.014 | 0.017 | −0.0052 |
| | (0.038) | (0.035) | (0.03) |
| D_{A} (mm²/s) | 0.000 | 0.018 | −0.0204 |
| | (0.047) | (0.043) | (0.0508) |
| D_{R} (mm²/s) | 0.021 | 0.017 | 0.0076 |
| | (0.04) | (0.037) | (0.0301) |
| Temporal | | | |
| stem | | | |
| Skewness | −0.002 | −0.016 | 0.0062 |
| | (0.047) | (0.047) | (0.0900) |
| FA | −0.015 | −0.013 | −0.0024 |
| | (0.028) | (0.029) | (0.0584) |
| MD (mm²/s) | 0.013 | 0.015 | −0.0009 |
| | (0.0028) | (0.03) | (0.024) |
| D_{A} (mm²/s) | 0.005 | 0.009 | −0.0036 |
| | (0.035) | (0.03) | (0.0267) |
| D_{R} (mm²/s) | 0.018 | 0.017 | 0.0021 |
| | (0.032) | (0.035) | (0.0371) |

TABLE 2D
Ranges of Coefficients and Asymmetry
Indices by Group and Hemisphere

| Superior Temporal Gyrus | | | Temporal Stem | | |
|---|---|---|---|---|---|
| SkewX | Left | | Right | Right | Right |
| AI | FA | D_{A} | MD | D_{A} | D_{R} |

TD

| | | | | | | |
|---|---|---|---|---|---|---|
| Minimum | −0.1008 | 0.2834 | 0.837 | 0.6554 | 0.9768 | 0.4684 |
| Maximum | 0.2731 | 0.3825 | 0.9384 | 0.7352 | 1.0789 | 0.5709 |

Autism

| | | | | | | |
|---|---|---|---|---|---|---|
| Minimum | −0.2327 | 0.269 | 0.8368 | 0.6669 | 0.9853 | 0.5029 |
| Max | 0.1614 | 0.3725 | 0.9745 | 0.7528 | 1.0732 | 0.6095 |

### C. Superior Temporal Gyrus

(i) Case-control tensor measure comparison. Table 3 contains ANCOVA-based tensor measures by hemisphere. FA and D_{A} were found to be equal bilaterally in the autism sample and greater on the left in the control sample. Conversely, D_{R} was greater on the left in the autism sample and equal bilaterally in the control sample. Between-group analysis identified a bilateral decrease of 4.27% in FA in autism (p=0.0064). Increases of 2.21% and 3.41% in mean MD and D_{R} in the autism sample were also observed (p=0.0248, p=0.0056). No between-group differences depended on participant age, hemisphere, age by hemisphere interaction, total brain volume, or white matter volume. In Table 3, the following apply: Symbol * indicates adjusted for total brain volume and performance IQ by repeated measures ANCOVA; symbol ^{†} indicates adjusted for total brain volume and performance IQ by repeated measures ANCOVA (no hemisphere by group interactions were statistically significant); symbol ^{‡} indicates cross-sectional age in years; symbol ^{§} indicates test for L−R=0 (e.g, symmetric or two-sided); symbol ** indicates all P-values corrected for multiple comparisons; and symbol ^{††} indicates not statistically significant at 0.05 and P-value greater than 0.20.

TABLE 3
STG Tensor Coefficient Means and Their Age-Related Changes

| | Typically Developing (n = 30) | | | | | | |
|---|---|---|---|---|---|---|---|
| | Hemisphere | | | | Age^{‡} (change/year) | | |
| | Left | Right | t^{§} | P** | Mean | t | P |
| FA | 0.3878 | 0.3755 | 3.83 | 0.0024 | 0.0024 | 3.99 | 0.0020 |
| MD (mm/s²) | 0.6507 | 0.6380 | 4.15 | 0.0012 | −0.0024 | −3.71 | 0.0040 |
| D_{A} (mm/s²) | 0.9581 | 0.9281 | 4.80 | <0.0001 | −0.0015 | −1.70 | n.s. |
| D_{R} (mm/s²) | 0.4970 | 0.4928 | 1.83 | n.s. | −0.0030 | −4.34 | 0.0008 |

| | Autism (n = 30) | | | | | | |
|---|---|---|---|---|---|---|---|
| | Hemisphere | | | | Age | | |
| | Left | Right | t | P | Mean | t | P |
| FA | 0.2906 | 0.2911 | — | n.s^{††} | 0.0014 | 2.35 | 0.1076 |
| MD (mm/s²) | 0.6725 | 0.6631 | 4.83 | <0.0001 | −0.0030 | −4.20 | 0.0011 |
| D_{A} (mm/s²) | 0.8754 | 0.8638 | 2.12 | 0.1716 | −0.0027 | −3.16 | 0.0158 |
| D_{R} (mm/s²) | 0.5712 | 0.5630 | 4.62 | 0.0008 | −0.0031 | −4.11 | 0.0014 |

| | Combined Between-Group Comparison | | | |
|---|---|---|---|---|
| | Group | | Group by Age | |
| | t | P | t | P |
| FA | −3.34 | 0.0015 | 1.50 | n.s. |
| MD (mm/s²) | 2.83 | 0.0063 | −0.28 | n.s. |
| D_{A} (mm/s²) | 1.89 | n.s. | −0.93 | n.s. |
| D_{R} (mm/s²) | 3.34 | 0.0015 | −0.14 | n.s. |

(ii) Age-related changes. Fractional anisotropy (FA) was age-invariant in the autism sample and increased with cross-sectional age in the control sample. Autism and control MD, D_{A}, and D_{R} decreased with age at equal rates.

(iii) Hemispheric asymmetry and its changes with cross-sectional age. Table 4 contains ANCOVA-adjusted asymmetry indices and age-related effects on tensor measures. The model-adjusted asymmetry indices are more symmetric in autism and more leftward in controls (e.g., typically developing control subjects) than their raw counterparts (see, e.g., Table 2). Between-group analysis showed that FA was atypically symmetric in autism, reduced by 105.4% of typical left-lateralization (p=0.0344). Fractional anisotropy asymmetry appeared stable with age in both samples. In autism, MD and D_{A} asymmetries exhibited atypical increases with cross-sectional age of 10.56% and 16.26% per year (P=0.0097 and P=0.0146), respectively. No additional group or age-related differences were found. In Table 4, the following apply: symbol * indicates adjusted for total brain volume and performance IQ by repeated measures ANCOVA; symbol ^{†} indicates adjusted for total brain volume and performance IQ by repeated measures ANCOVA (no hemisphere by group interactions were statistically significant); symbol ^{‡} indicates η=2(L−R)/(L+R), wherein hemispheric asymmetry found if η is significantly different from 0; symbol ^{§} indicates cross-sectional age in years; symbol ** indicates all P-values corrected for multiple comparisons; and symbol ^{††} indicates not statistically significant at 0.05 and P-value greater than 0.20.

TABLE 4
STG Hemisphere Asymmetry Indices
and Their Age-Related Changes
Control (n = 30)

| | Asymmetry Index | | | Age (change/year)^{§} | | |
|---|---|---|---|---|---|---|
| | Mean | t | P** | Mean | t | P |
| FA | 0.0373 | 4.00 | 0.0019 | −0.0037 | −2.01 | n.s^{.††} |
| MD (mm/s²) | 0.0194 | 4.23 | 0.0010 | −0.0013 | −1.48 | n.s. |
| D_{A} (mm/s²) | 0.0336 | 5.03 | 0.0001 | −0.0025 | −1.88 | n.s. |
| D_{R} (mm/s²) | 0.0074 | 1.72 | n.s. | −0.0004 | −0.49 | n.s. |

Autism (n = 30)

| | Asymmetry Index | | | Age (change/year) | | |
|---|---|---|---|---|---|---|
| | Mean | t | P | Mean | t | P |
| FA | −0.0024 | −0.24 | n.s. | 0.0031 | 1.63 | n.s. |
| MD (mm/s²) | 0.0143 | 6.13 | <0.00001 | 0.0017 | 3.93 | 0.0023 |
| D_{A} (mm/s²) | 0.0134 | 2.58 | 0.0642 | 0.0028 | 2.90 | 0.0301 |
| D_{R} (mm/s²) | 0.0150 | 4.34 | 0.0008 | 0.0008 | 1.19 | n.s. |

Combined Between-Group Comparison^{†}

| | |
|---|---|
| Group | Group by Age |

| | t | P | t | P |
|---|---|---|---|---|
| FA | −2.73 | 0.0344 | 2.22 | 0.1216 |
| MD (mm/s²) | −0.96 | n.s. | 3.18 | 0.0097 |
| D_{A} (mm/s²) | −2.23 | 0.1208 | 3.04 | 0.0146 |
| D_{R} (mm/s²) | 1.32 | n.s. | 1.41 | n.s. |

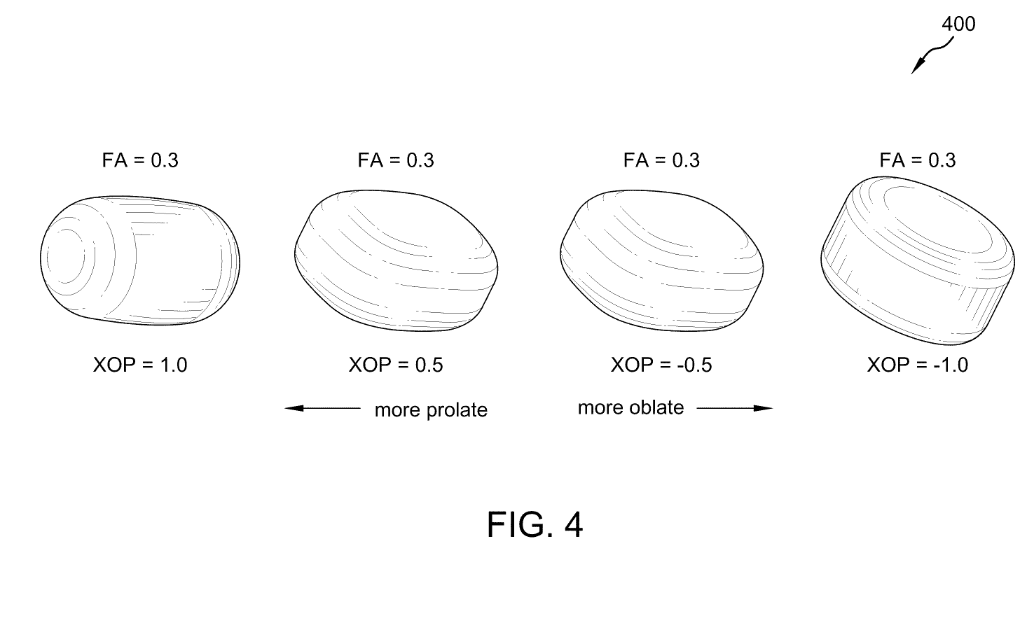

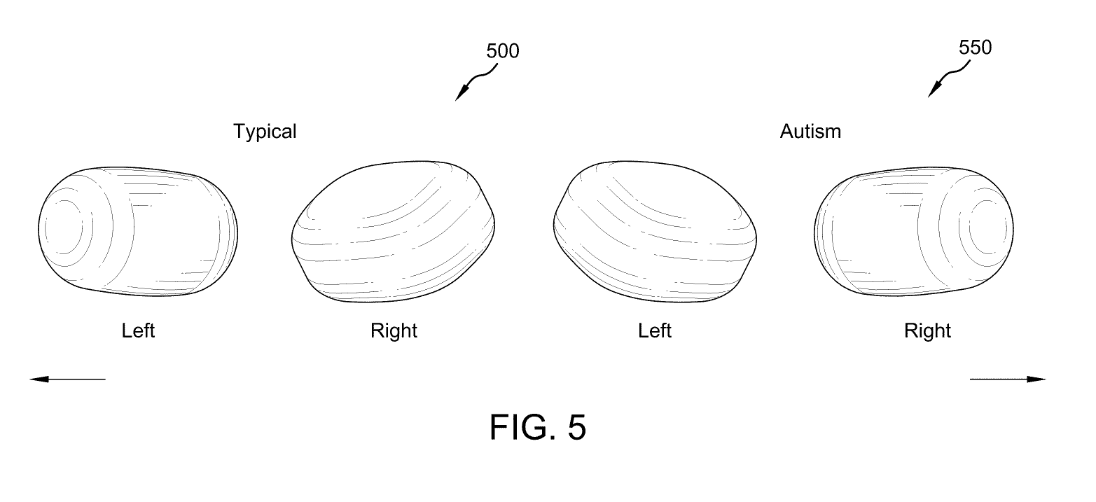

(iv). Tensor skewness. In the STG, tensor skewness asymmetry (referred to and labeled as SkewX) was greater (e.g., more coherent, more prolate) on the left in control subjects but greater on the right in autism subjects; in an aspect, control left=0.5172, control right=0.5022; and autism left=0.5053, autism right=0.5173; with P=0.044). Such hemispheric reversal (see, e.g., FIG. 5) indicates an atypical loss of directional diffusion coherence in autism in the left STG, which atypical loss occurs in addition to any atypicality in FA that can occur in such neurological disorder. FIG. 4 presents a diagram 400 that depicts example prolate versus oblate tensor shapes for values of tensor skewness asymmetryc (SkewX) near values characteristic of a sampling (or sample) of subjects (e.g., sample of control subjects). Such tensor shapes have been generated at the same fractional anisotropy (FA) value of 0.3, which also is close to group values. As illustrated in FIG. 4, tensor shape differences can be subtle in this range. However, as illustrated in diagrams 500 and 550 in FIG. 5, the hemispheric asymmetry of STG tensor skewness in autism, however, exhibited a more significant reversal of its typical left lateralization when accounting simultaneously for the effects of FA in the STG. For instance, asymmetry indices: −0.0221 in autism vs. 0.0303 in control, with P=0.0199. Tensor skewness was unaffected by cross-sectional age in both groups.

### D. Temporal Stem

Most between-group differences found in tensor measures were qualitatively identical to those found in the STG but of lesser magnitude and statistical significance. Temporal stem FA asymmetry and age-related changes in MD and D_{A} were unaffected.

### E. Tensor Skewness Asymmetry (SkewX), Group Discrimination. and Classification

In an aspect, tensor skewness asymmetry is the most or substantially the most salient measure in the multivariate classification algorithm. The group discrimination rule determined from the first sample indicated that the following six tensor measures possessed 91.6% accuracy; 93.6% sensitivity; 89.6% specificity; 90% positive predictive value; 87.5% negative predictive value; and 83.3% reliability: (1) STG tensor skewness asymmetry (SkewX), (2) left STG FA, (3) right TS D_{A}, (4) right TS D_{R}, (5) right TS MD, and (6) STG D_{A}. (See Table 5).

In Table 5, the following apply: the hemispheric asymmetry index is 2× (Left−Right)/(Left+Right) and hemispheric asymmetry found if significantly different from 0; symbol ^{†} indicates positive predictive value; symbol ^{‡} indicates negative predictive value; and symbol ^{§} indicates intraclass correlative coefficient, equivalent to Cohen's κ.

TABLE 5
Group Discrimination and Classification of the Aggregated Diffusion
Tensor Metrics With and Without tensor skewness asymmetry (SkewX)

| | Training Sample | | Replication Sample | |
|---|---|---|---|---|
| | Autism | Control | Autism | Control |
| | (n = 30) | (n = 30) | (n = 12) | (n = 7) |
| | With | Without | With | Without |
| | Tensor | Tensor | Tensor | Tensor |
| | Skewness | Skewness | Skewness | Skewness |
| | Asymmetry* | Asymmetry | Asymmetry | Asymmetry |
| Accuracy (%) | 91.6 | 85.6 | 94.7 | 68.4 |
| Sensitivity (%) | 93.6 | 85.9 | 91.7 | 66.7 |
| Specificity (%) | 89.6 | 85.2 | 100 | 71.4 |
| PPV (%)^{†} | 90.0 | 65.6 | 100 | 80.0 |
| NPV (%)^{‡} | 93.3 | 71.5 | 87.5 | 55.6 |
| Reliability (%)^{§} | 83.3 | 68.9 | 89.0 | 36.0 |

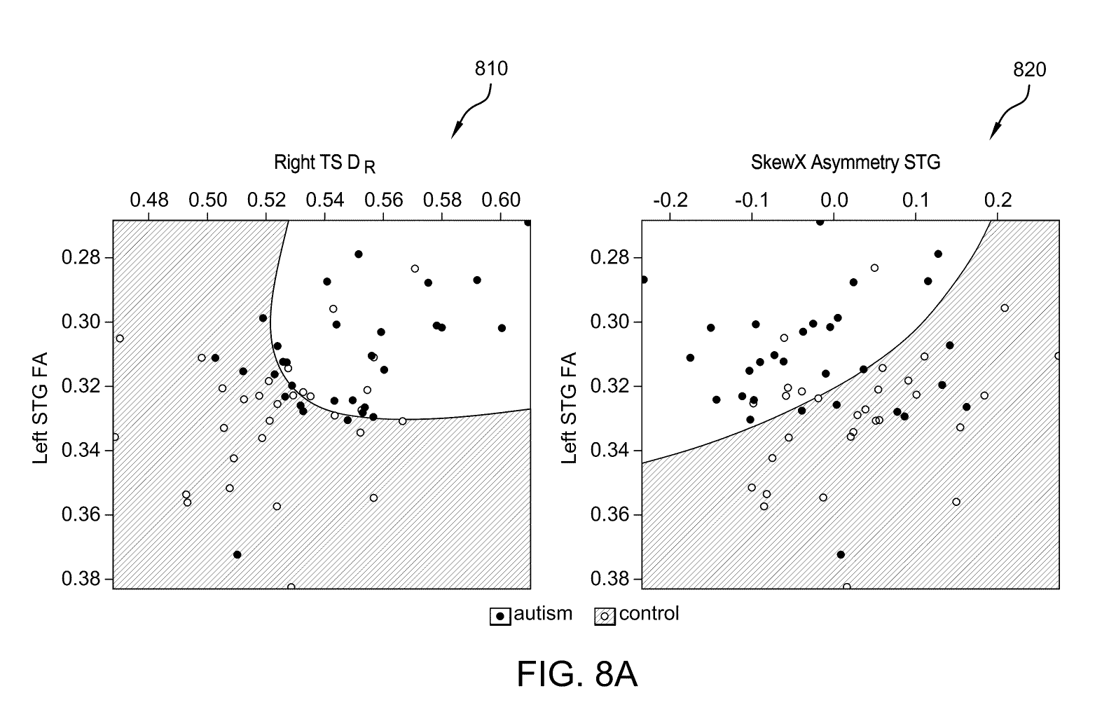

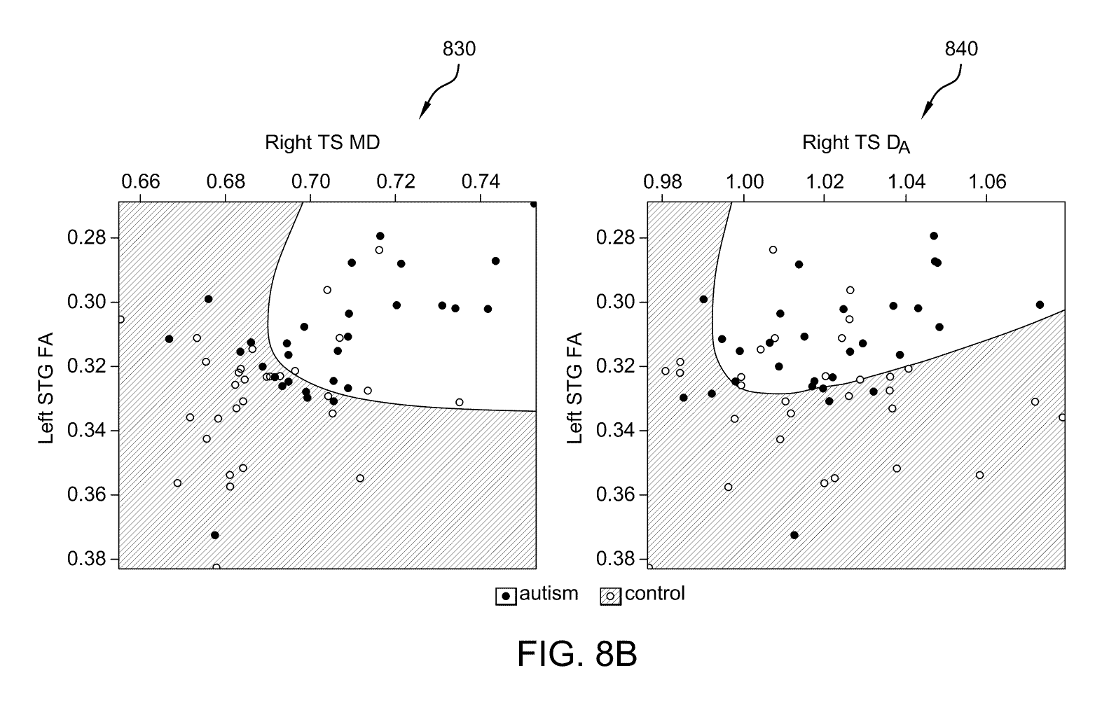

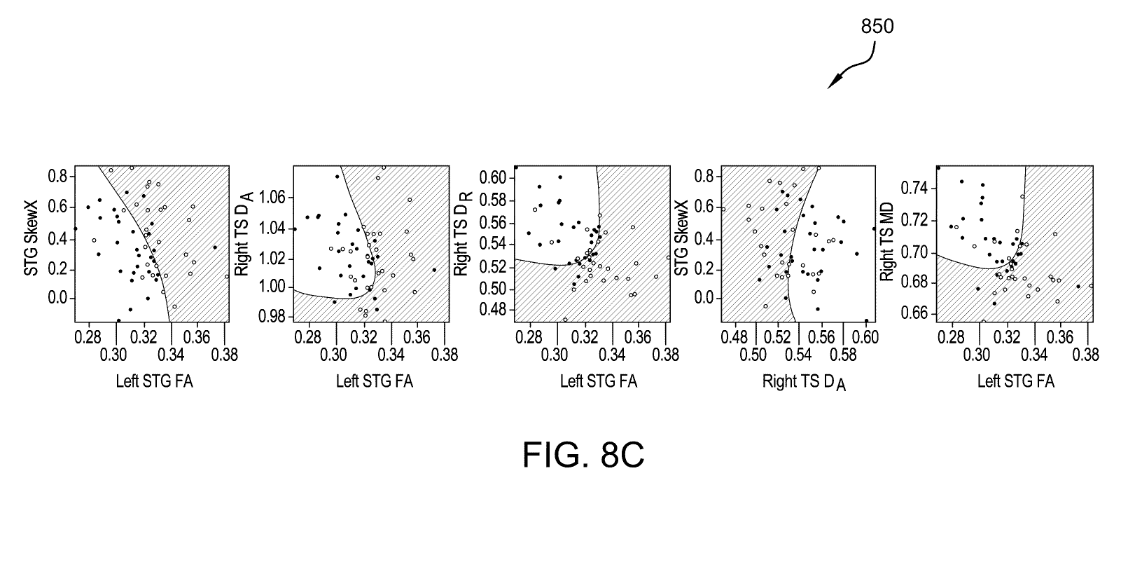

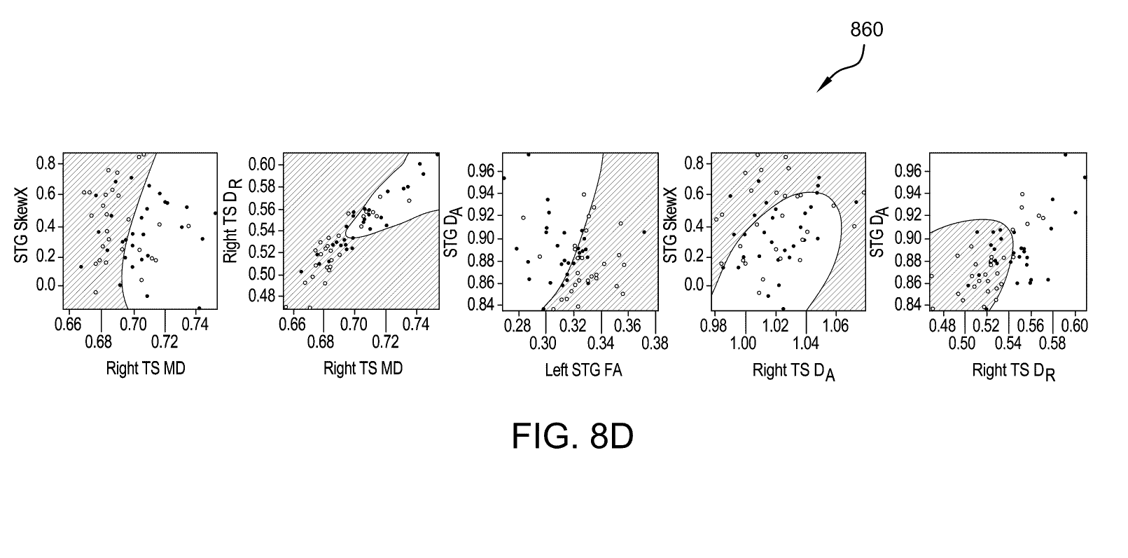

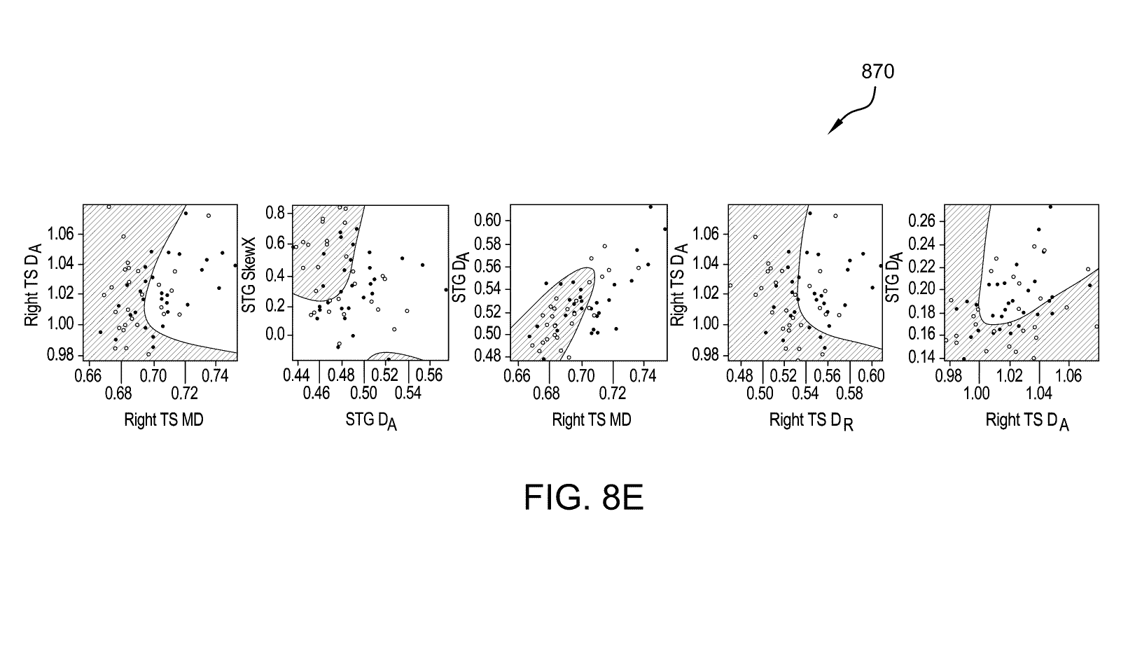

FIGS. 8A-8E depict objective group classification boundaries for the four pairs of tensor measures associated with the highest classification ability among all 15 pairs. An objective group classification boundary also is referred to as a decision boundary. Pair-wise representations of objective classification boundaries illustrated in diagrams 710-770 are projections onto a bi-dimensional sub-space of a multivariate hypersphere in a Q-dimensional (Q is a natural number) sub-space of features or attributes, wherein such hypersphere includes a Q-dimensional hyperplance representative of a decision boundary in the sub-space of features. In an aspect, features or attributes that span the Q-dimensional sub-space of features or attributes include at least one group of diffusion tensor metrics. Selection of Q and the related group of classification features or classification attributes can be based at least on (i) clinical data related to a neurological disease or neurological disorder and (ii) anatomical characteristics of at least one central nervous system (CNS) structure that can be afflicted by the neurologic disease or the neurological disorder. As an example, for Alzheimer's Disease, the at least one CNS structure can comprise the brian cortex, hippocampus (e.g., anterior, para-hippocampal regions), precuneus, or amygdala. As another example, for epilepsy the at least one CNS structure can comprise the brain cortex (e.g., temporal lobes, limbic part of the temporal pole, entorhinal cortex), hippocampus, or amygdala. As yet another example, for aphasia the at least one CNS structure can comprise Broca's area (e.g., frontal lobe) or Wernicke's area (e.g., temporal lobe) in the brain. As a further example, for Parkinson's disease the at least one CNS structure can comprise the substantia nigra. As a still further example, for schizophrenia the at least one CNS structure can comprise the STG, middle temporal gyrus, anterior cingulate, amygdale, frontal and parietal lobes, hippocampus, or prefrontal cortex. In an alternative or additional example, for Wilson's disease the at least one CNS structure can comprise basal ganglia (e.g., putamen and globus pallidus), while for obsessive compulsive disorders the at least one CNS structure can comprise frontal lobes (e.g., lateral orbitofrontal cortex), basal ganglia, or cingulum. In a further alternative or additional example, for attention-deficit hyperactivity disorder (ADHD) the at least one CNS structure can comprise frontal lobes (e.g., dorsal prefrontal cortex), temporal lobes (e.g., anterior temporal lobe), caudate nucleus, or cerebellum.

At least one of (a) the parameter Q or (b) a related set of classification features (e.g., diffusion tensor metric(s)) can control, at least in part, group classification performance such as accuracy, sensitivity, specificity, reliability, or the like. As an example, Q=6 and the group of diffusion metrics can be include (I) superior temporal gyrus (STG) tensor skewness asymmetry, (II) left STG fractional anisotropy, (III) right temporal stem (TS) axial diffusivity, (IV) right TS radial diffusivity, (V) right TS mean diffusivity, and (VI) STG axial diffusitivity. For such sub-space of classification attributes, in an example embodiment, classification performance is illustrated in Table 5.

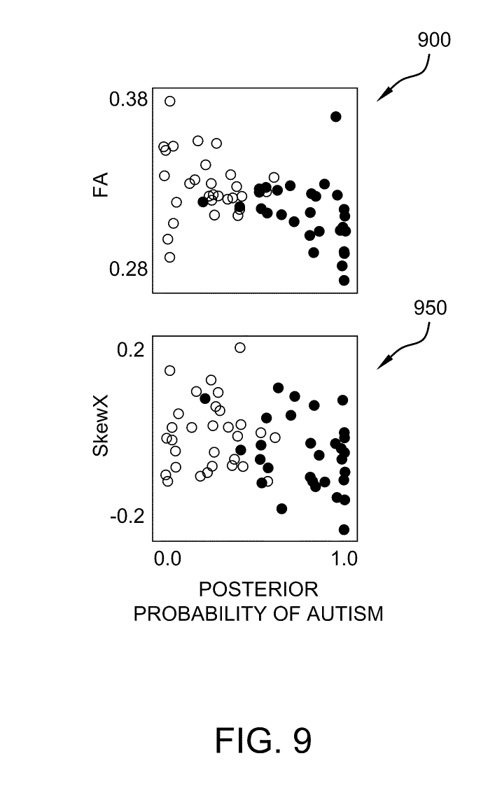

Application of a multivariate classifier, such as a discrimination rule embodied in a discrimation function resultant from QDA, generated with classifier attributes (1)-(6) to a second independent replication sample yields 94.7% accuracy; 91.7% sensitivity (11/12 subjects); 100% specificity (7/7 subjects); 100% positive predictive value (7/7 subjects); 87.5% negative predictive value (7/8 subjects); and 89.0% reliability. As depicted in FIG. 9, STG tensor skewness asymmetry (also referred to as SkewX) accounts for the largest increment accomplished in classification ability, see diagram 950; left STG FA also provides increment in classification ability but to a lesser extent than SkewX, see diagram 900. It should be appreciated that, as described hereinbefore, the second independent replication sample serves to assess performance in a non-empty set of data that have not been utilized to generate the discrimation rule, or multivariate classifer. In an example embodiment, a set of diffusion tensor metrics that defines a five-dimensional sub-space of classification attributes and comprises (2) left STG fractional anisotropy, (3) right temporal stem (TS) axial diffusivity, (4) right TS radial diffusivity, (5) right TS mean diffusivity, and (6) STG axial diffusitivity, can be utilized to generate a discrimination rule to classify presence or absence of autism in accordance with aspects described herein. The so-generated discrimination rule, or multivariate classifier, does not rely on STG tensor skewness asymmetry as a classification feature. As illustrated in Table 6, such discrimination rule applied to the second independent replication sample yields 68.4% accuracy (13/19 subjects); 66.7% sensitivity (8/12 subjects); 71.5% specificity (5/7 subjects); 80.0% positive predictive value (8/10 subjects); 55.6% negative predictive value (5/9 subjects); and 36.0% reliability. Thus, in an aspect, application of the discrimination rule generated without STG tensor skewness asymmetry provides a discrimination algorithm that is deficient when compared to the discrimination rule generated with inclusion of STG tensor skewness asymmetry as a classification attribute.

In an example embodiment, a support vector machine that employs the six tensor metrics (I)-(VI) provided substantially degraded classification performance. In an aspect, such SVM supplied unacceptable magnitudes of training accuracy (e.g., 86.7%), positive predictive value (e.g., 80.5%), and reliability (e.g., 63.2%). Such comparison demonstrates an example advantage of fitting a model, such as quadratic discrimination in an example embodiment, when assumptions of the machine-learning model hold.

Furthermore, in an aspect, a novel DTI-based signature, or biomarkerm, can be determined for autism. As shown in Table 6, the novel DTI-based signature comprises: (1) low SkewX, e.g., decreased directional diffusion coherence in the left hemisphere, and high parallel diffusion (e.g., high D_{A}) in the STG, and (2) high isotropic diffusion (e.g., high MD), high perpendicular diffusion (e.g., high D_{R}), and low parallel diffusion (e.g,. low D_{A}) in the right hemispheric TS.

TABLE 6
A Novel DTI Signature for Autism

| | STG | TS |
|---|---|---|
| LOW | SkewX | |
| | Left FA | |
| HIGH | D_{A} | Right MD |
| | | Right D_{R} |
| | | Right D_{A} |

### F. Clinical Correlation

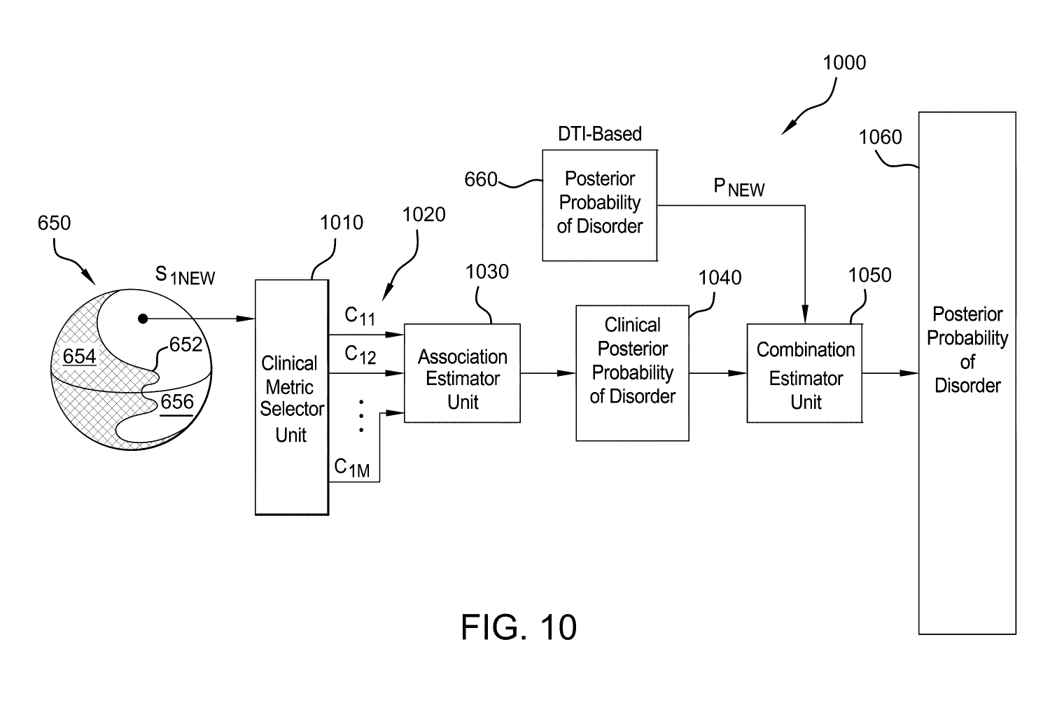

FIG. 10 illustrates an example system 1000 that enables integration of imaging-based prediction of a neurological disease or neurological disorder with clinical prediction thereof in accordance with aspects described herein. In example system 1000, a clinical metric selector unit 1010 (also referred to as clinical metric selector 1010) can receive a set 1020 of one or more clinical metrics {C₁₁, C₁₂, . . . C_{1B}} pertinent to a test subject S_{1New} who a multivariate classifier assigns to the class (e.g., class 1) of subjects having a neurological disease or neurological disorder. In certain embodiments, clinical metric selector 1010 can receive at least one clinical metric from data storage 130. Clinical metric selector unit 1010 can supply at least one of the clinical metrics {C₁₁, C₁₂, . . . C_{1B}} to an association estimator unit 1030 that, in an aspect, can assign a weight (a real number, a vector of real numbers, etc.) each clinical metrics that is received from the clinical metric selector 1030. In addition, based at least on a group of weights assigned to one or more clinical metrics and a group of rules (e.g., a predefined function), association estimator unit 1030 can construct a probability distribution of a test subject having the neurological disease or neurological disorder and evaluate a clinical posterior probability of disorder 1040. In example system 1000, a combination estimator unit 1050 receives a DTI-based posterior probability of disorder 660 (labeled P_{New}) and the clinical probability of disorder 1040, and can combine such probabilities to issue a combined posterior probabilituy of disorder (CPPD) 1060. Combination estimator unit 1050 can apply a combination rule to the received probabilities and, in response to application of the combination rule, issue CPPD 1060. As an example, the combination rule can assign a first weight w₁ (e.g., a real number) to the clinical probability of disorder 1040 and a second weight w₂ (e.g., a real number) to P_{New} 660, and issue CPPD as the weigthed combination of such probabilities. In an aspect, the weights can be dynamic and thus, vary over time; dynamic weights can accommodate, in part, historical changes in the set of one or more clinical metrics {C₁₁, C₁₂, . . . C_{1B}} associated with the test subject S_{1New}.

Other aspects of integration of clinical assessment of a neurological disease or a neurological disorder includes can comprise correlation of individual sets of diffusion tensor metrics that define a sub-space of classification features with at least one clinical metric (e.g., C₁₁ such as SRS). Additional or alternative quantities that can be correlated with at least one clinical metric include, but are not limited to, distance of at least one diffusion tensor metric to a decision boundary.

### G. Comorbidity and Medication Status

Three combinations of comorbidity and medication status in the autism sample were observed: 53% both (n=16), 33% neither (n=10), 10% comorbidity only (n=3), and 3% medication only (n=1). Comorbidity, which generally is highly correlated with medication use (Kendall's τ=0.73), exhibited a significant association with temporal stem D_{A} asymmetry primarily (t=2.22, P=0.035, uncorrected).

### H. Language Function and Social Responsiveness

White matter microstructure asymmetry in the temporal stem primarily or, in certain embodiments, exclusively, affects CELF-3 scores. In an example embodiment, in the autism sample, average CELF-3 total scores improved by 6 points for every 0.01 unit increase in leftward MD asymmetry in the temporal stem (t=2.76, P=0.014) compared to a negligible 0.2 point increase in the control sample (also referred to herein as typicall developing subject sample). In an aspect, all tensor metrics other than leftward MD asymmetry were unaffected. SRS was also unaffected.

### J. White Matter Microstructure Asymmetry, and Age-Related Changes in Typical Development

In the typically developing control subjects (also referred to as either typically developing controls or controls), FA increases while MD and D_{R} decrease with cross-sectional age in the STG bilaterally. In an aspect, leftward asymmetry of FA, MD, and D_{A} is age-invariant, whereas D_{R} appears to symmetric and age-invariance. Such aspect indicates maturation of STG white matter microstructure between late childhood and early adulthood with increasing anisotropy and stable asymmetry. Mean FA and MD in the left (L) and right (R) STG are similar in a group of 22 older normal controls (mean 40.4, range 18-55 years of age). In another aspect, a leftward yet statistically insignificant FA increase is found in 6 normal adults. It should be appreciated that conventionally, white matter microstructure asymmetry of the TS in typically developing subjects has not been studied or it has been studied marginally. The temporal stem is one of the main efferent and afferent white matter “bridges” that connect the anterior temporal lobe to the frontal lobe and thalamus. The TS contains the uncinate and inferior occipitofrontal fasciculi, the posterior limbs of the anterior commissure, and the inferior thalamic radiations and Meyer's loop of the visual system. Conventional study(ies) that examined the uncinate and occipitofrontal fasciculi in healthy elderly persons have found symmetric FA and MD. Such study(ies) found that temporal stem FA is highly lateralized to the left, MD is symmetric, D_{A} is left-lateralized, and D_{R} is lateralized to the right, indicating that TS white matter microstructure is mature and asymmetric by early childhood in typically developing subjects.

### K. Atypical Asymmetry and Additional White Matter Microstructure Deviations in Autism

A decrease is present in the spatial organization of fibers in the left STG in autism: STG tensor skewness is atypically more oblate, or incoherent, on the left and more prolate, or coherent, on the right. Such deviation of tensor skewness is accompanied by the loss of typical leftward FA asymmetry in the autistic STG. Since D_{A} is unaffected in the STG and TS, the observed MD increases are likely due to increases in D_{R}. Dysmyelination may thus be implicated if other related factors such as crossing fibers, fiber packing, intracellular viscosity, osmotic pressure, and neurofibrils (such as protein content) may be ruled out. Some or all of these factors may be affected in autism. Data and related results produced with one or more embodiments of the subject disclosure suggest reduced spatial organization of white matter fibers and possibly dysmyelination in the autistic STG.

### L. Tensor Skewness Asymmetry (SkewX) and White Microstructure-Based Group Discrimination

It should be appreciated that in conventional systems or through conventional processes tensor skewness asymmetry (SkewX) has not been investigated in autism. The aggregate analysis of white matter microstructure atypicalities elides artificially separated biological factors and can provide a more comprehensive interpretation of the multiple facets of atypical brain circuitry exhibited in autism and other neurological disorders. Tensor skewness asymmetry appears to possess unique, doubly-discriminating properties which provide at least tensorial discrimination and the hemispheric discrimination. When combined with additional atypical tensor metrics, tensor skewness asymmetry in the superior temporal gyrus (STG) can play a highly influential role in the neuropathology of autism by dramatically increasing the accuracy, sensitivity, sensitivity, and reliability of the biological discrimination (or classification) of subjects with and without autism.

### M. Age-Related Changes in White Matter Microstructure and its Asymmetry in Autism

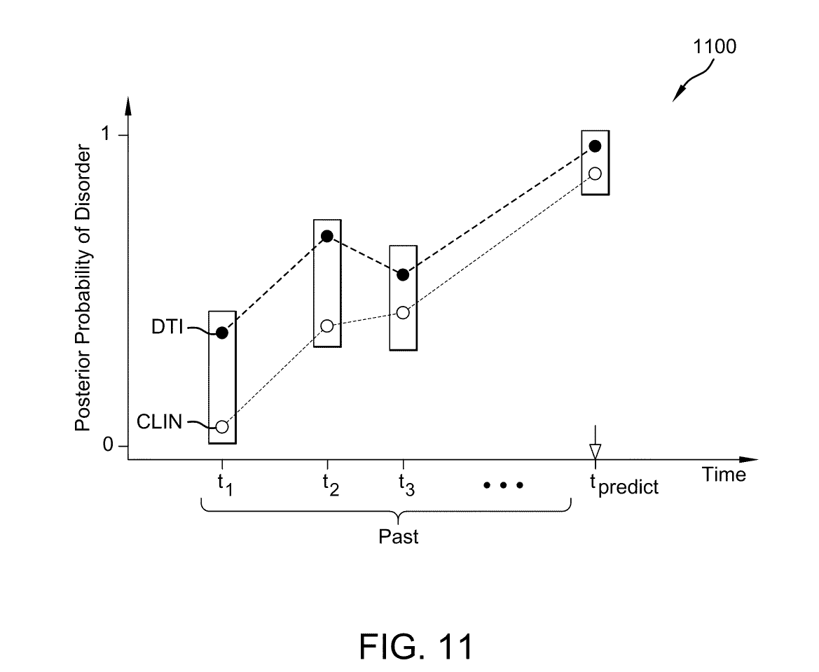

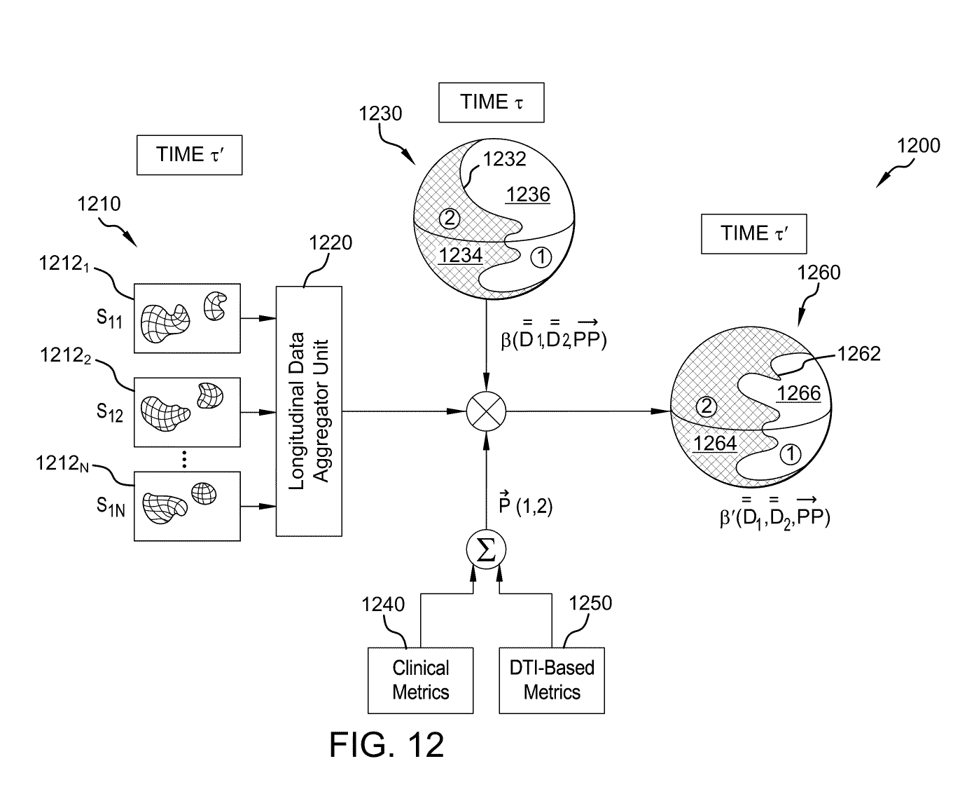

In certain aspects, cross-sectional age-related changes in STG FA, MD, and D_{R} are similar to changes identified in typically developing control subjects. Yet, age-related variations in STG MD and D_{A} asymmetry differed. In the subject disclosure, the reduced leftward asymmetry of STG MD and D_{A} in autism becomes more leftward with cross-sectional age and approaches values more consistent with those of typically developing control subjects. Such “normalization” can serve to improve proximal and distal neuronal connectivity. As illustrated in FIG. 11, such normalization leads to more consistent results regarding prediction of presence of autism. In diagram 1100, a sketch of longitudinal results for the posterior probability of disorder (PPD) illustrates a reduction amongst such probability when extracted in test subjects in accordance with aspects of the subject disclosure (see, e.g., FIG. 6) and the PPD that arises entirely from clinical assessment—solid circles represent PPD based on imaging data (labeled DTI) related to WMM and related hemispheric asymmetries, whereas open circles represent PPD extracted from clinical (CLIN) assessment(s). An imaging-based probability of disorder can change as a function of time as a result of change(s) in a decision boundary associated with a multivariate classifier calculated in accordance with aspects decribes herein. In an aspect, as time progresses, determination (e.g., calculation) of the multivariate classifier for a current time (e.g., τ′) can utilize at least a portion of input data from a determination (e.g., calculation) of a multivariate classifier at a prior time (e.g., τ). As an example, as illustrated in example system 1200 in FIG. 12, for a group 1210 of N subjects at current time (e.g., τ′), imaging data at the current time can be collected (e.g., via imaging unit 110) and processed (formatted, aggregated, etc.) through a longitudinal data aggregator unit 1220 (also referred to as longitudinal data aggregator 1220. Prior probabilities of disorder determined through a combination of clinical metrics 1240 and imaging-based metrics 1250 (e.g., a set of diffusion tensor metrics) can be associated to a set of imaging data, such as diffusion tensor imaging data and related diffusion tensor metrics. A machine-learning model that has yielded, at a past time (e.g., τ), a decision boundary 1232 defining a first class 1236 and a second class 1234 can be utilized to compute an updated decision boundary 1262 at the current time (e.g., τ′).

The updated decision boundary 1262 separates a first updated class 1264 and a second updated class 1266, wherein the first updated class 1264 can correspond to a time-evolved version of the first class 1234 and, likewise, the second updated class 1266 can correspond to a time-evolved version of the second class 1236. In an embodiment, the updated decision boundary 1262 can supply a PPD at the current time (e.g., τ′) that is more consistent with a PPD at a past time (e.g., τ) and based on clinical observations, see, e.g., FIG. 11; such increase in consistency can originate from utilization of PPD(s) determined in prior instance(s) and based on clinical metrics (e.g., clinical metrics 1240) and imaging-based metrics (e.g., DTI-based metrics 1250) determined in prior times. In another embodiment, such consistency can originate from change(s) in WMM which can be assessed directly from imaging data observed at the current time (e.g., τ′) for a group 1210 of subjects previously imaged (e.g., the group 1210 of subjects can be the same group of subjects {S₁₁, S₁₂ . . . S_{1N}} imaged at time τ). In such a scenario, longitudinal data aggregator unit 1220 can generate an updated non-empty set of training imagining data which can be employed to learn the updated decision boundary 1262. In certain aspects, longitudinal data aggregator unit 1220 can generate a library of a plurality of non-empty sets of training imaging data.

In certain embodiments, availability of longitudinal imaging data enables generation of larger sub-space(s) of classification features with respect to a sub-space of classification features employed for at least one prior instant. Such generation can be “variational” in that at least one classification feature (Φ), such as a diffusion tensor metric, added to a first sub-space of classification features at a first time (e.g., τ) to generate a larger, second sub-space of classification features at a second time (e.g., τ′) can be constructed (e.g., computed) as a function F(·) of a difference amongst a classification feature (f) evaluated at the second time (e.g., f=φ′) and the classification feature evaluated at the first time (f=φ); namely: Φ=F(φ′−φ), wherein F is a configurable function. As an example, f can be SkewX and F(x)=x, which yields a variational classification feature defined as SkewX(t=τ′)−SkewX(τ). A group of functions {F} that define variational classification features cam be retained in data storage 130. In an aspect, longitudinal data aggregator unit 1220 can generate (e.g., compute) the larger, second sub-space of classification features based on at least one function F₀ in the {F}.

Tables 7-10 present data related to diffusion tensor metrics at disparate times, e.g., longitudinal data, for an autism group of subject and for a normal group of subject. Variations (Δ) defined as the difference amongst a diffusion tensor metric (e.g., SkewX) at a prior (or past) time and a current time are also presented in such Tables. Time-dependent diffusion tensor metrics are determined from imaging data collected at two disparate times in accordance with aspects described herein.

TABLE 7
Diffusion tensor metrics for STG for a group of autism subjects for a
current time and variations (Δ) of the illustrated metrics.

| SkewX | Δ | FA (Left) | Δ | D_{A} (Left) | Δ |
|---|---|---|---|---|---|
| −1.1309971 | 1.15034153 | 0.347416 | −0.156058 | 0.882524 | 0.203083 |
| 0.08336039 | −0.1092338 | 0.388027 | −0.122471 | 0.94446 | 0.210999 |
| −10.620961 | 10.3407459 | 0.363592 | −0.192643 | 0.893003 | 0.193409 |
| 0.13192696 | −0.5316101 | 0.274747 | −0.094651 | 0.9107535 | 0.3028965 |
| 1.04292237 | −0.9897334 | 0.30186 | −0.096767 | 0.995739 | 0.081469 |
| −0.2342706 | 0.58644742 | 0.298897 | −0.082148 | 0.884022 | 0.3238855 |
| −1.4383247 | 0.92548045 | 0.280518 | −0.080304 | 0.977849 | 0.1447205 |
| 2.75473969 | −2.8039395 | 0.342803 | −0.120092 | 0.9117025 | 0.256622 |

TABLE 8
Diffusion tensor metrics for STG for a group of control subjects for a current
time and variation (Δ) of the diffusion tensor metrics with respect to a prior time.

| SkewX | Δ | FA (Left) | Δ | D_{A} (Left) | Δ |
|---|---|---|---|---|---|
| 0.55624684 | −0.6428876 | 0.207245 | 0.025359 | 1.0202375 | 0.0365505 |
| 0.9428562 | −1.0606306 | 0.2103 | 0.01873 | 1.1545765 | 0.0548775 |
| −0.0917137 | 0.30274967 | 0.185578 | 0.025682 | 1.038956 | 0.05851 |
| −1.1746448 | 1.00222474 | 0.331184 | −0.097854 | 0.9487815 | 0.165347 |
| −0.1491421 | −0.2571575 | 0.194141 | 0.027549 | 1.052109 | 0.0491605 |
| 0.36128403 | −0.1903395 | 0.214289 | 0.018696 | 1.0558485 | 0.0024485 |

TABLE 9
Diffusion tensor metrics for Right TS for subjects in autism group
for a current time and variation (Δ) of the diffusion
tensor metrics with respect to a prior time.

| MD | Δ | D_{A} | Δ | D_{R} | Δ |
|---|---|---|---|---|---|
| 0.675177 | 0.199462 | 0.985261 | 0.227523 | 0.520135 | 0.387027 |
| 0.676443 | 0.205526 | 0.997596 | 0.248763 | 0.515867 | 0.401979 |
| 0.684507 | 0.189639 | 0.994 | 0.177489 | 0.52976 | 0.37202 |
| 0.713442 | 0.30175 | 0.971256 | 0.331642 | 0.584535 | 0.456062 |
| 0.764458 | 0.154116 | 1.061905 | 0.178747 | 0.615735 | 0.335961 |
| 0.708374 | 0.224531 | 0.990986 | 0.258186 | 0.567068 | 0.39572 |
| 0.745823 | 0.165471 | 1.026747 | 0.209602 | 0.605361 | 0.336438 |
| 0.68416 | 0.284754 | 1.000895 | 0.283539 | 0.585171 | 0.402273 |

TABLE 10
for diffusion tensor metrics for Right TS for subjects in
a group of control subjects for a current time and variation (Δ)
of the diffusion tensor metrics with respect to a prior time.

| MD | Δ | D_{A} | Δ | D_{R} | Δ |
|---|---|---|---|---|---|
| 0.854011 | 0.03212 | 1.191114 | 0.050417 | 0.88628 | 0.03085 |
| 0.976633 | −0.00095 | 1.316073 | −0.016936 | 1.006972 | −0.008987 |
| 0.880847 | −0.034757 | 1.208792 | 0.004726 | 0.909173 | −0.026685 |
| 0.734581 | 0.148729 | 1.026121 | 0.216852 | 0.588811 | 0.327339 |
| 0.906247 | 0.0301 | 1.235006 | 0.03221 | 0.936813 | 0.031178 |
| 0.922655 | −0.031992 | 1.267264 | −0.017896 | 0.950836 | −0.025987 |

### N. Psychiatric Comorbidity, Psychotropic Medications, and White Matter Microstructure Asymmetry in Autism

Conventional systems or protocols have rarely examined associations of comorbidity and psychotropic medication with DT-MRI measurements, and mainly in schizophrenia. Analysis of imaging data of corpus callosum conveys that medicated participants with autism are three (3) times more likely than their unmedicated counterparts to have more normalized callosal microstructure; namely, higher FA and lower MD (P=0.006, one-sided). In the subject disclosure, subjects with autism who have had a lifetime history of comorbid psychopathology and have been taking psychotropic medication(s) at the time of evaluation through one or more embodiments described herein present more typical leftward asymmetry of D_{A} in the TS. Such finding indicates that the clinical benefits of medication can be mediated in part by improvements in the microstructural integrity of white matter and its inter-hemispheric organization. Prospective longitudinal studies are needed to determine if better white matter microstructure precedes or is the result of medication treatment.

### O. Language Functioning and White Matter Microstructure Asymmetry in Autism

Typical language functioning in the STG, which includes very early auditory processing of language-like pre-lexical stimuli, is initially symmetric and becomes increasingly lateralized leftward with development. Damage to the TS is known to result in abnormalities of verbal functions, including verbal declarative memory and learning, in addition to spatial and visual deficits. In control samples, or groups of typically developing control subjects, in the subject disclosure, symmetries in TS MD and D_{R} are positively correlated with a measure of verbal memory (e.g., WSR-Scaled). The latter feature is consistent with a similar finding in the arcuate fasciculus. Such findings in the control findings convey that increased leftward MD asymmetry in the TS can be associated with improved language functioning in autism. In an aspect, these increases appear to be driven mainly by a leftward increase in TS D_{A} and can be associated with improved language functioning due to more organized, less tortuous fibers.

Thus data and related results of the subject disclosure suggest or demonstrate several features. (A) The atypicality of inter-hemispheric organization in white matter microstructure of the STG and TS in the autistic sample was determined. The most salient white matter microstructure deviation observed in the autism sample is the atypical hemispheric reversal of diffusion tensor skewness in the superior temporal gyrus (STG). Tensor skewness and its hemispheric asymmetry are additional components of directional diffusion coherence not captured by fractional anisotropy (FA). The hemispheric reversal of tensor skewness in autism indicates that directional diffusion along white matter fibers in the STG is less coherent on the left and more coherent on the right relative to healthy populations. (B) The directional diffusion coherence reversal in autism also is accompanied by a symmetrizing loss of typical leftward asymmetry and a bilateral decrease in FA in the STG. (C) Local diffusion in all directions (MD) and particularly in directions perpendicular to its primary direction (D_{R}) are elevated in the STG, indicating increased fiber crossing, dysmyelination, or other divergent white matter microstructure biology found in the disorder. The leftward STG asymmetry of the local parallel diffusion component (D_{A}) also is greatly reduced in autism. (D) Subjects with autism exhibit atypical age-related increases in the leftward asymmetry of multi-directional and parallel diffusion in STG white matter. (E) In the temporal stem of autistics, directional coherence (FA) is reduced while mean multi-directional and perpendicular diffusion are elevated in autism. In addition, a tendency towards typical leftward asymmetry of mean parallel diffusion in the temporal stem of those psychiatrically comorbid subjects with autism taking psychotropic medications. (F) Three white matter microstructure (e.g., tensor skewness asymmetry, left hemisphere FA, and bilateral D_{A}) deviations in the superior temporal gyrus and three WMM (e.g., D_{A}, D_{R}, and MD) deviations in the right temporal stem can be useful biological indicators (or biomarkers) of autism. In certain embodiments, these six atypical diffusion tensor metrics possess substantially high ability to discriminate between subjects with autism and subjects without autism in the training sample and the replication sample with 93% accuracy and sensitivity, and 90% specificity.

These results in the subject disclosure indicate that six abnormalities of white matter microstructure in the superior temporal gyrus and temporal stem, especially in the novel tensor asymmetry index, are a biological marker for autism in similar subjects with autism (see, e.g., Table 6). In certain embodiments, the objective DTI-based signature comprises low SkewX, decreased directional diffusion coherence in left hemisphere superior temporal gyrus and high superior temporal gyrus parallel diffusion, and high isotropic diffusion, high perpendicular diffusion and low parallel diffusion in right hemisphere temporal stem. The objective DTI-based biomarker displays classification performance that is superior to any or most any other conventional biological measurements.

In an aspect, the various embodiments of the subject disclosure can be implemented in hardware or software, or a combination of both. Several aspects or features of the subject disclosure can be implemented in a computer program using standard programming techniques following the method steps and figures described herein.

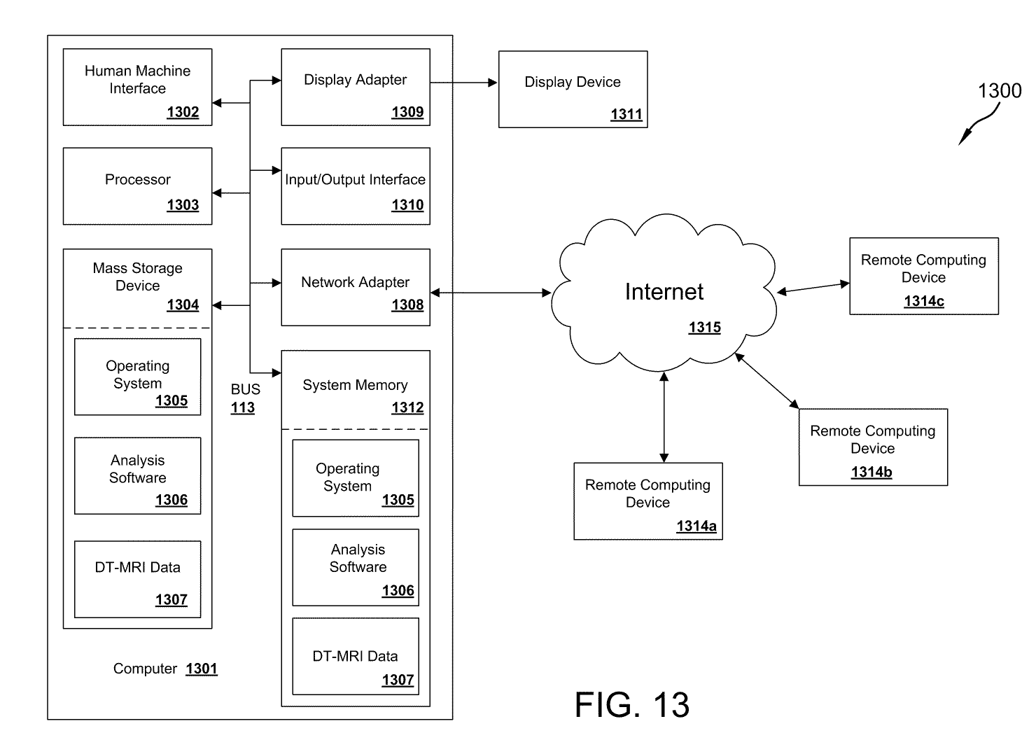

The system has been described above as comprised of units (see, e.g., FIG. 6, FIG. 10, FIG. 12). One skilled in the art will appreciate that this is a functional description and that the respective functions can be performed by software, hardware, or a combination of software and hardware. A unit can be software, hardware, or a combination of software and hardware. The units can comprise the Analysis Software 1306 as illustrated in FIG. 13 and described. Analysis software 1306 can include one or more set of computer-executable code instructions that, in response to execution by at least one processor, such as processor 1303, can cause the at least one processor or a unit comprising the at least one processor to carry out the various processes or methods described in the subject disclosure. In one example aspect, an individual unit can comprise a computer 1301 as illustrated in FIG. 13 and described below. In another aspect, a group of one or more units can comprise the computer 1301. In yet another aspect, computer 1301 can embody a single unit or a group of units.

FIG. 13 illustrated a block diagram of an example operating environment 1300 that enables various features of the subject disclosure and performance of the various methods disclosed herein. This exemplary operating environment is only an example of an operating environment and is not intended to suggest any limitation as to the scope of use or functionality of operating environment architecture. Neither should the operating environment be interpreted as having any dependency or requirement relating to any one or combination of components illustrated in the exemplary operating environment.

The various embodiments of the subject disclosure can be operational with numerous other general purpose or special purpose computing system environments or configurations. Examples of well known computing systems, environments, and/or configurations that can be suitable for use with the systems and methods comprise, but are not limited to, personal computers, server computers, laptop devices, and multiprocessor systems. Additional examples comprise set top boxes, programmable consumer electronics, network PCs, minicomputers, mainframe computers, distributed computing environments that comprise any of the above systems or devices, and the like.

The processing effected in the disclosed systems and methods can be performed by software components. The disclosed systems and methods can be described in the general context of computer-executable instructions, such as program modules, being executed by one or more computers or other devices. Generally, program modules comprise computer code, routines, programs, objects, components, data structures, etc. that perform particular tasks or implement particular abstract data types. The disclosed methods also can be practiced in grid-based and distributed computing environments where tasks are performed by remote processing devices that are linked through a communications network. In a distributed computing environment, program modules can be located in both local and remote computer storage media including memory storage devices.

Further, one skilled in the art will appreciate that the systems and methods disclosed herein can be implemented via a general-purpose computing device in the form of a computer 1301. The components of the computer 1301 can comprise, but are not limited to, one or more processors or processing units 1303, a system memory 1312, and a system bus 1313 that couples various system components including the processor 1303 to the system memory 1312. In the case of multiple processing units 1303, the system can utilize parallel computing.

The system bus 1313 represents one or more of several possible types of bus structures, including a memory bus or memory controller, a peripheral bus, an accelerated graphics port, and a processor or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCI-Express bus, a Personal Computer Memory Card Industry Association (PCMCIA), Universal Serial Bus (USB) and the like. The bus 1313, and all buses specified in this description also can be implemented over a wired or wireless network connection and each of the subsystems, including the processor 1303, a mass storage device 1304, an operating system 1305, Analysis Software 1306, DT-MRI data 1307, a network adapter 1308, system memory 1312, an Input/Output Interface 1310, a display adapter 1309, a display device 1311, and a human machine interface 1302, can be contained within one or more remote computing devices 1314a,b,c at physically separate locations, connected through buses of this form, in effect implementing a fully distributed system.

The computer 1301 typically comprises a variety of computer readable media. Exemplary readable media can be any available media that is accessible by the computer 1301 and comprises, for example and not meant to be limiting, both volatile and non-volatile media, removable and non-removable media. The system memory 1312 comprises computer readable media in the form of volatile memory, such as random access memory (RAM), and/or non-volatile memory, such as read only memory (ROM). The system memory 1312 typically contains data such as DT-MRI data 1307 and/or program modules such as operating system 1305 and Analysis Software 1306 that are immediately accessible to and/or are presently operated on by the processing unit 1303.

In another aspect, the computer 1301 also can comprise other removable/non-removable, volatile/non-volatile computer storage media. By way of example, FIG. 13 illustrates a mass storage device 1304 which can provide non-volatile storage of computer code, computer readable instructions, data structures, program modules, and other data for the computer 1301. For example and not meant to be limiting, a mass storage device 1304 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 1304, including by way of example, an operating system 1305 Analysis Software 1306. Each of the operating system 1305 and Analysis Software 1306 (or some combination thereof) can comprise elements of the programming and the Analysis Software 1306. DT-MRI data 1307 also can be stored on the mass storage device 1304. DT-MRI data 1307 can be stored in any of one or more databases known in the art. Examples of such databases comprise, DB2®, Microsoft® Access, Microsoft® SQL Server, Oracle®, mySQL, PostgreSQL, and the like. The databases can be centralized or distributed across multiple systems.

In another aspect, the user can enter commands and information into the computer 1301 via an input device (not shown). Examples of such input devices comprise, but are not limited to, a keyboard, pointing device (e.g., a “mouse”), a microphone, a joystick, a scanner, tactile input devices such as gloves, and other body coverings, and the like. These and other input devices can be connected to the processing unit 1303 via a human machine interface 1302 that is coupled to the system bus 1313, but can be connected by other interface and bus structures, such as a parallel port, game port, an IEEE 1394 Port (also known as a Firewire port), a serial port, or a universal serial bus (USB).

In yet another aspect, a display device 1311 also can be connected to the system bus 1313 via an interface, such as a display adapter 1309. It is contemplated that the computer 1301 can have more than one display adapter 1309 and the computer 1301 can have more than one display device 1311. For example, a display device can be a monitor, an LCD (Liquid Crystal Display), or a projector. In addition to the display device 1311, other output peripheral devices can comprise components such as speakers (not shown) and a printer (not shown) which can be connected to the computer 1301 via Input/Output Interface 1310. Any step and/or result of the methods can be output in any form to an output device. Such output can be any form of visual representation, including, but not limited to, textual, graphical, animation, audio, tactile, and the like.

The computer 1301 can operate in a networked environment using logical connections to one or more remote computing devices 1314a,b,c. By way of example, a remote computing device can be a personal computer, portable computer, a server, a router, a network computer, a peer device or other common network node, and so on. Logical connections between the computer 1301 and a remote computing device 1314a,b,c can be made via a local area network (LAN) and a general wide area network (WAN). Such network connections can be through a network adapter 1308. A network adapter 1308 can be implemented in both wired and wireless environments. Such networking environments are conventional and commonplace in offices, enterprise-wide computer networks, intranets, and the Internet 1315.

As an illustration, application programs and other executable program components such as the operating system 1305 are illustrated herein as discrete blocks, although it is recognized that such programs and components reside at various times in different storage components of the computing device 1301, and are executed by the data processor(s) of the computer. An implementation of Analysis Software 1306 can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” comprise volatile and non-volatile, removable and non-removable media implemented in any methods or technology for storage of information such as computer readable instructions, data structures, program modules, or other data. Exemplary computer storage media comprises, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

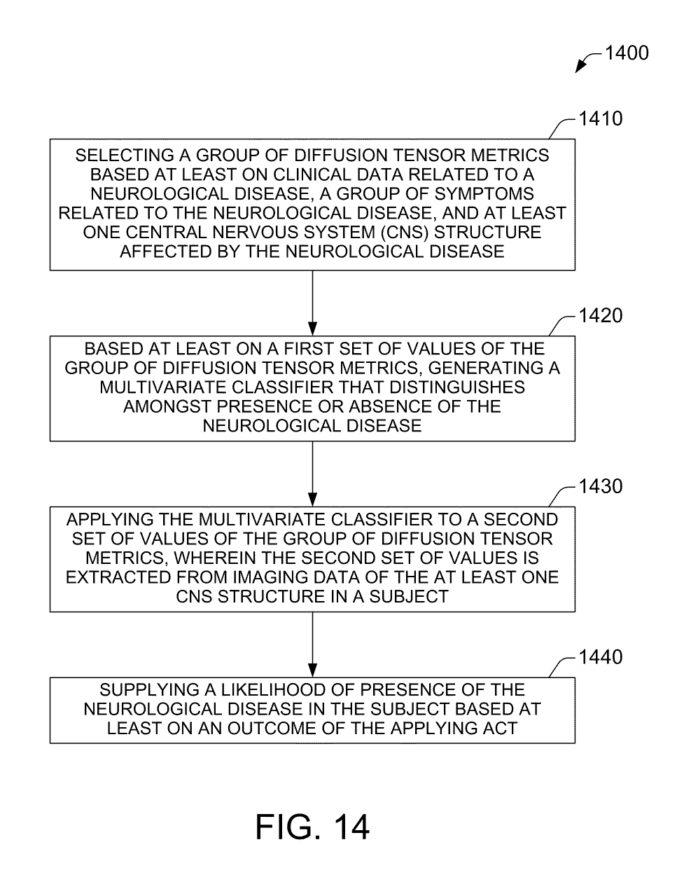

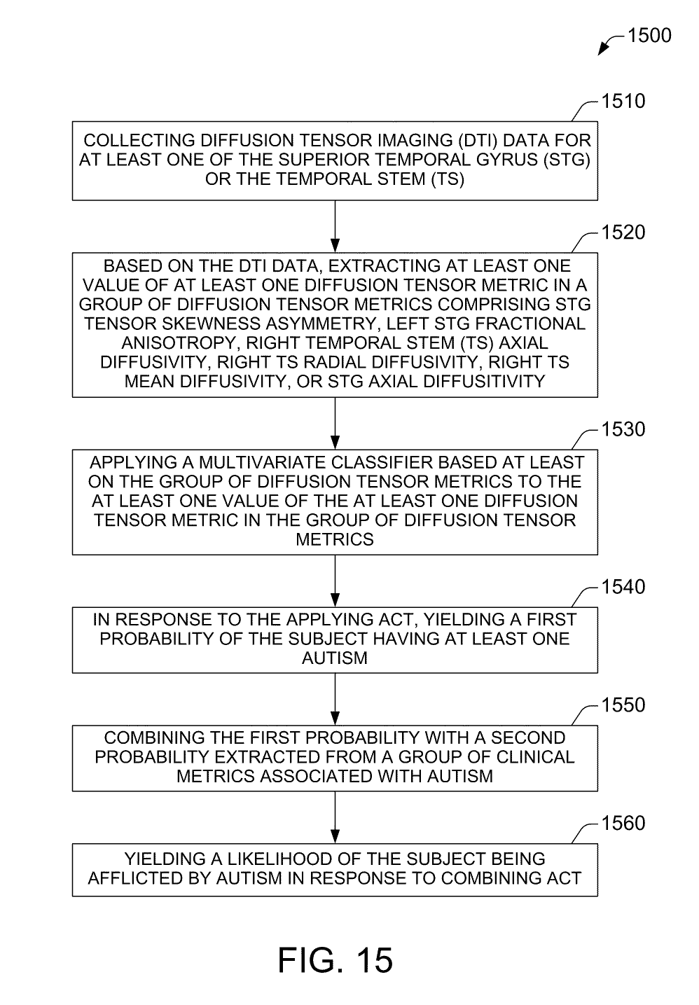

In view of the various embodiments of system(s), apparatus(es), device(s), and the like described herein, processes or methods that can be performed by such systems(s), apparatus(es), device(s) and the like, can be better appreciated in connection with flowcharts of FIGS. 14-15. It should be appreciated that while the subject methods are represented as flowcharts, other suitable representation also are possible. Acts in the described method(s) and any process(es) described herein can be performed in different order from the one illustrated in FIGS. 14-15. In addition, in certan embodiments, two or more of the described acts can be performed simultaneously or substantially simultaneously. FIGS. 14-15 present flowcharts of example methods 1400 and 1500 for identifying a neurological disease in accordance with aspects described herein. Regarding example method 1400, act 1410 comprises selecting a group of diffusion tensor metrics based at least on clinical data related to a neurological disease, a group of symptoms related to the neurological disease, and at least one central nervous system (CNS) structure affected by the neurological disease. Act 1420, comprises generating a multivariate classifier that distinguishes amongst presence or absence of the neurological disease. In an aspect, the subject act is performed based at least on a first set of values of the group of diffusion tensor metrics as described hereinbefore. Act 1430, comprises applying the multivariate classifier to a second set of values of the group of diffusion tensor metrics, wherein the second set of values is extracted from imaging data of the at least one CNS structure in a subject. Act 1440, comprises supplying a likelihood of presence of the neurological disease in the subject based at least on an outcome of the applying act. In an aspect, the outcome of the applying act comprises a posterior probability of presence of the neurological disease in the subject.

In certain embodiments, the example method 1400 comprises reiterating act 1410 (e.g., the selecting act) and act 1420 (e.g., the generating act) in response to a classification performance score being below a performance threshold. In an aspect, the classification performance score can be a composite index including, for instance, one or more of classification sensitivity, classification specificity, classification accuracy, classification reliability, negative predictive value (NPV), or positive predictive value (PPV). Performance threshold(s) can be configurable and can be defined statically or dynamically. Moreover, the reiterating act 1410 reiterating acts 1410 and act 1420 can comprise reiterating such acts (e.g., the selecting act and the generating act) for a plurality of two or more occasions, wherein the selecting act can comprise computing at least one variational diffusion tensor metric based at least on a difference amongst a diffusion tensor metric in the group determined at a first occasion in the plurality and the diffusion tensor metric determined at a second occasion in the plurality.

Example method 1500 illustrates an example method for identification of autism. Act 1510 comprises collecting diffusion tensor imaging (DTI) data for at least one of the superior temporal gyrus (STG) or the temporal stem (TS). Based on the DTI data collected at act 1510, act 1520 comprises extracting at least one value of at least one diffusion tensor metric in a group of diffusion tensor metrics comprising STG tensor skewness asymmetry, left STG fractional anisotropy, right temporal stem (TS) axial diffusivity, right TS radial diffusivity, right TS mean diffusivity, or STG axial diffusitivity. Act 1530 comprises applying a multivariate classifier based at least on the group of diffusion tensor metrics to the at least one value of the at least one diffusion tensor metric in the group of diffusion tensor metrics. Act 1540 comprises yielding a first probability of the subject having at least one autism in response to act 1530 (e.g., the applying act) and at least one result thereof. Act 1550 comprises combining the first probability with a second probability extracted from a group of clinical metrics associated with autism. Act 1560 comprises yielding a likelihood of the subject being afflicted by autism in response to act 1550 (e.g., the combining act) and at least one result thereof.

In certain embodiments, computer 1301 can be configured to perform or can perform the example methods 1400 and 1500. In alternative or additional embodiments, one or more units described in the various embodiments of the subject specification can be configured to perform or can perform examples methods 1400 and 1500.

The systems and methods of the subject disclosure can employ artificial intelligence (AI) techniques such as machine learning and iterative learning. Examples of such techniques include, but are not limited to, expert systems, case based reasoning, Bayesian networks, behavior based AI, neural networks, fuzzy systems, evolutionary computation (e.g., genetic algorithms), swarm intelligence (e.g., ant algorithms), and hybrid intelligent systems (e.g., Expert inference rules generated through a neural network or production rules from statistical learning).

While the systems, devices, apparatuses, protocols, processes, and methods have been described in connection with example embodiments and specific illustrations, it is not intended that the scope be limited to the particular embodiments set forth, as the embodiments herein are intended in all respects to be illustrative rather than restrictive.

Unless otherwise expressly stated, it is in no way intended that any protocol, procedure, process, or method set forth herein be construed as requiring that its acts or steps be performed in a specific order. Accordingly, where a process or method claim does not actually recite an order to be followed by its acts or steps or it is not otherwise specifically recited in the claims or descriptions of the subject disclosure that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification or annexed drawings, or the like.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims or inventive concepts.

What is claimed is:

1. A method comprising:
selecting a group of diffusion tensor metrics based at least on clinical data related to a neurological disease, a group of symptoms related to the neurological disease, and at least one central nervous system (CNS) structure affected by the neurological disease;
based at least on a first set of values of the group of diffusion tensor metrics, generating a multivariate classifier that distinguishes amongst presence or absence of the neurological disease;
applying the multivariate classifier to a second set of values of the group of diffusion tensor metrics, wherein the second set of values is extracted from imaging data of the at least one CNS structure in a subject; and
supplying a likelihood of presence of the neurological disease in the subject based at least on an outcome of the applying act.

2. The method of claim 1, wherein the outcome of the applying act comprises a posterior probability of presence of the neurological disease in the subject.

3. The method of claim 1, further comprising reiterating the selecting act and the generating act in response to a classification performance score being below a performance threshold.

4. The method of claim 1, further comprising reiterating the selecting act and the generating act for a plurality of two or more occasions, wherein the selecting comprises
computing at least one variational diffusion tensor metric based at least on a difference amongst a diffusion tensor metric in the group determined at a first occasion in the plurality and the diffusion tensor metric determined at a second occasion in the plurality.

5. The method of claim 2, wherein the supplying act comprises supplying the likelihood of presence of at least one of autism, an autism spectrum disorder, Fragile X, seizure, aphasia, Parkinson's disease, Wilson's disease, amyotrophic lateral sclerosis, tuberous sclerosis, Alzheimer's disease, coma, epilepsy, stroke, depression, multiple sclerosis, schizophrenia, addiction, neurogenic pain, cognitive/memory dysfunction, obsessive compulsive disorder (OCD), attention-deficit hyperactivity disorder (ADHD), dementia, traumatic brain injury, post-traumatic stress disorder (PTSD), a minimally conscious or vegetative state, locked-in syndrome, spinal cord injury, peripheral neuropathy, migraine, epilepsy, a brain tumor, or a spinal tumor.

6. The method of claim 1, further comprising extracting at least one value of the second set of values of the group of diffusion tensor metrics from diffusion tensor imaging data of the at least one CNS structure in the subject.

7. The method of claim 1, wherein the selecting act comprises selecting one or more of superior temporal gyrus (STG) tensor skewness asymmetry, left STG fractional anisotropy, right temporal stem (TS) axial diffusivity, right TS radial diffusivity, right TS mean diffusivity, or STG axial diffusitivity.

8. The method of claim 7, wherein the supplying act comprises supplying the likelihood of presence of at least one of autism or an autism spectrum disorder.

9. The method of claim 1, further comprising mapping white matter microstructure of the at least one CNS structure based at least in part on at least one value of the group of diffusion tensor metrics.

10. The method of claim 1, the acts further comprising mapping the hemispheric organization of white matter of the the at least one CNS structure based at least in part on at least one value of the group of diffusion tensor metrics.

11. The method of claim 1, further comprising treating the subject based at least on supplying a likelihood of presence of the neurological disease.

12. The method of claim 11, further comprising evaluating the efficacy of treatment administered to the subject via the treating act, wherein the evaluating comprises
determining for the subject a first value of a first diffusion tensor metric in the group of diffusion tensor metrics; and
comparing the first value of the first diffusion tensor metric in the group of diffusion tensor metrics to a second value of a second diffusion tensor metric for a control subject, wherein
establishing the efficacy of the treatment by repeating the determining act and the comparing act at least one time.

13. The method of claim 11, wherein the establishing act comprises establishing the efficacy of treatment by repeating the determining act and comparing act at scheduled intervals.

14. The method of claim 9, wherein the mapping comprises generating at least one diffusion tensor image of white matter microstructure of the at least one CNS structure for the subject being in utero, an infant, a child, an adolescent, or an adult.

15. A system comprising:
a memory that retains data and logic; and
a processor functionally coupled to the memory and programmed by the logic to
receive imaging data related to nervous tissue in at least one central nervous system (CNS) structure of a subject;
extract from the imaging data a set of one or more values of a group of diffusion tensor metrics comprising tensor skewness asymmetry; and
apply a multivariate classifier based at least on the group of diffusion tensor metrics comprising tensor skewness asymmetry to the set of one or more values; and
in response to application of the multivariate classifier, yield a probability of the subject having a neurological disease or neurological disorder that affects the at least one CNS structure.

16. The system of claim 15, wherein the processor is further programmed by the logic to deliver the probability of the subject having the neurological disease or neurological disorder.

17. The system of claim 15, wherein the subject is in utero, a child, an adolescent, or an adult.

18. The system of claim 15, wherein the imaging data comprises diffusion tensor imaging data.

19. A method comprising:
collecting diffusion tensor imaging (DTI) data for at least one of the superior temporal gyrus (STG) or the temporal stem (TS);
based on the DTI data, extracting at least one value of at least one diffusion tensor metric in a group of diffusion tensor metrics comprising STG tensor skewness asymmetry, left STG fractional anisotropy, right temporal stem (TS) axial diffusivity, right TS radial diffusivity, right TS mean diffusivity, or STG axial diffusitivity;
applying a multivariate classifier based at least on the group of diffusion tensor metrics to the at least one value of the at least one diffusion tensor metric in the group of diffusion tensor metrics; and
in response to the applying act, yielding a first probability of the subject having at least one autism.

20. The method of claim 19, further comprising
combining the first probability with a second probability extracted from a group of clinical metrics associated with autism; and
in response to the combining act, yielding a likelihood of the subject being afflicted by autism.
